(19) United States
(12) Patent Application Publication
(10) Pub. No.: US 2025/0297954 A1
(43) Pub. Date: Sep. 25, 2025

(54) METHOD OF MONITORING PARAMETERS IN TURBID SOLUTIONS

(71) Applicant: CSL BEHRING AG, Bern (CH)

(72) Inventors: Michael BIERI, Bern (CH); Tobias HECK, Bern (CH)

(73) Assignee: CSL BEHRING AG, Bern (CH)

(21) Appl. No.: 18/878,312

(22) PCT Filed: Jun. 28, 2023
(86) PCT No.: PCT/EP2023/067708

§ 371 Date: Dec. 23, 2024

(30) Foreign Application Priority Data
Jun. 28, 2022 (AU) 2022901797
Dec. 19, 2022 (AU) 2022903894

(51) Int. Cl. G01N 21/359; G01N 21/27; G01N 33/487

(52) U.S. Cl. CPC G01N 21/359; G01N 21/274; G01N 33/487; G01N 2201/1293

(57) ABSTRACT

The invention relates to methods for determining the concentration of an analyte in a sample obtained from processing of blood-derived plasma, the method comprising applying a light source in the near-infrared spectrum to a test sample obtained from processing of blood-derived plasma; measuring reflectance, transmission or transflectance of the test sample over a range of near-infrared wavelengths, thereby generating test wavelength spectra, comparing the test wavelength spectra with reference wavelength spectra obtained from reference samples having known concentrations of the analyte, to determine the concentration of the analyte in the sample. The invention also relates to the development of a multivariate model for use in determining the concentration of an analyte, for example, total protein or alcohol (e.g. ethanol).

### CROSS REFERENCE TO EARLIER APPLICATIONS

This application is the U.S. National Stage of International Application No. PCT/EP2023/067708, filed Jun. 28, 2023, and claims priority from Australian provisional applications 2022901797, filed Jun. 28, 2022, and 2022903894, filed Dec. 19, 2022, the entire contents of which are each hereby incorporated by reference.

### FIELD OF THE INVENTION

The invention relates to methods for in-line, at-line, off-line and/or on-line monitoring of parameters in turbid solutions or suspensions and the application of same in methods for purifying solutions comprising proteins and other components.

### BACKGROUND OF THE INVENTION

The demand for purified proteins such as specific antibodies has increased considerably. Such purified proteins can be used for therapeutic and/or diagnostic purposes.

Human blood plasma has been industrially utilized for decades for the production of widely established and accepted plasma-protein products such as human albumin (HSA), immunoglobulin (IgG), clotting factor concentrates (clotting Factor VIII, clotting Factor IX, prothrombin complex etc.) and inhibitors (antithrombin, C1-inhibitor etc.). In the course of the development of such plasma-derived drugs, plasma fractionation methods have been established, leading to intermediate products enriched in certain protein fractions, which then serve as the starting composition for plasma-protein product/s. Typical processes are reviewed in e.g. Molecular Biology of Human Proteins (Schultze H. E., Heremans J. F.; Volume I: Nature and Metabolism of Extracellular Proteins 1966, Elsevier Publishing Company; p. 236-317). These kinds of separation technologies allow for the production of several therapeutic plasma-protein products from the same plasma donor pool. This is economically advantageous over producing only one plasma-protein product from one donor pool, and has therefore been adopted as the industrial standard in blood plasma fractionation.

One example of this type of fractionation process, cold ethanol fractionation of plasma, was pioneered by E. J Cohn and his team during World War II, primarily for the purification of albumin (Cohn E J, et al. 1946, J. Am. Chem. Soc. 62: 459-475). The Cohn fractionation process involves increasing the ethanol concentration in stages, from 0% to 40%, while lowering the pH from neutral (pH 7) to about 4.8, resulting in the precipitation of albumin. Whilst Cohn fractionation has evolved over the past 70 years or so, most commercial plasma fractionation processes are based on the original process or a variation thereof (e.g. Kistler/Nitschmann), exploiting differences in pH, ionic strength, solvent polarity and alcohol concentration to separate plasma into a series of major precipitated protein fractions (such as Fractions I to V in Cohn).

Variations to the Cohn Fractionation process have been developed with the aim of improving polyvalent IgG recovery. For example Oncley and co-workers used Cohn Fractions II+III as a starting material with different combinations of cold ethanol, pH, temperature and protein concentration to those described by Cohn, to produce an active immune globulin serum fraction (Oncley et al., (1949) J. Am. Chem. Soc. 71, 541-550). Today, the Oncley method is the classic method used for production of polyvalent IgG. Nevertheless, it is known that approximately 5% of gamma-globulins (antibody-rich portion) is co-precipitated with Fraction I and about 15% of the total gamma-globulin present in plasma is lost by the Fraction II+III step (See Table III, Cohn E J, et al. 1946, J. Am. Chem. Soc. 62: 459-475). The Kistler/Nitschmann method aimed to improve IgG recovery by reducing the ethanol content of some of the precipitation steps (Precipitation B vs Fraction III). The increased yield, however, is at the expense of the purity (Kistler & Nitschmann, (1962) Vox Sang. 7, 414-424).

Initially, immunoglobulin G (IgG) preparations derived from these fractionation processes were successfully used for the prophylaxis and treatment of various infectious diseases. However as ethanol fractionation is a relatively crude process the IgG products contained impurities and aggregates to an extent that they could only be administered intramuscularly. Since that time additional improvements in the purification processes have led to IgG preparations suitable for intravenous (called IVIg) and subcutaneous (called SCIg) administration.

It has been estimated that approximately 30 million liters of plasma were processed worldwide in 2010, providing a range of therapeutic products including about 500 tonnes of albumin and 100 tonnes of IVIg. The IVIg market accounts for about 40-50% of the entire plasma fractionation market (P. Robert, Worldwide supply and demand of plasma and plasma derived medicines (2011) J. Blood and Cancer, 3, 111-120). Thus, with demands for IVIg remaining strong (along with increasing demands for SCIg) there remains a need to improve immunoglobulin recoveries from plasma and related fractions. Preferably, this must be achieved in a way that ensures the recovery of other plasma derived therapeutic proteins is not adversely affected.

From a commercial perspective, the initial fractionation processes are critical to the overall production time and costs associated with the production of a therapeutic protein, particularly plasma derived proteins, since the subsequent purification steps will depend on the yield and purity of the protein(s) of interest within these initial fractions. Whilst several variations of the cold ethanol fractionation process have been developed for plasma derived protein in order to improve protein yield at lower operating costs, higher protein yields are typically associated with lower purity.

There is a need for new and/or improved methods for determining the concentration of various analytes in complex solutions during plasma processing to improve downstream efficiency, reduction in waste and/or improve final product yield. Due to the heterogeneity of plasma-derived product solutions and suspensions, the quantification of key chemical components, such as protein, is complex and to date, has only be achieved by use of off-line analytical methods that require sampling effort and analysis lead times of commonly several days. Therefore, there is a need for new analytical processes to determine concentration of analytes in turbid, particularly highly turbid, solutions or suspensions.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method for determining the concentration of an analyte in a sample obtained from processing of blood-derived plasma, the method comprising:
applying a light source in the near-infrared spectrum to a test sample obtained from processing of blood-derived plasma;
measuring reflectance, transmission or transflectance of the test sample over a range of near-infrared wavelengths, thereby generating test wavelength spectra,
comparing the test wavelength spectra with reference wavelength spectra obtained from reference samples having known concentrations of the analyte, to determine the concentration of the analyte in the sample.

Typically, NIR-spectra contain hundreds of variables and therefore some form of multivariate data analysis method is preferably used to analyze raw data from the measurements. Such multivariate data analysis methods are well known in the art and includes Partial least squares regression (PLS); PLS Discriminant Analysis (PLS-DA); Ordinary Least Squares (OLS) regression; MLR (multiple linear regression); OPLS (Orthogonal-PLS); SVM (support vector machines); GLD (general discriminant analysis); GLMC (generalized linear model); GLZ (generalized linear and non-linear model); LDA (Linear Discriminant Analysis); classification trees; cluster analysis; neural networks; and Pearson correlation.

In one aspect, the present invention provides a method for determining the concentration of an analyte in a sample obtained from processing of blood-derived plasma, the method comprising:
applying a light source in the near-infrared spectrum to a test sample obtained from processing of blood-derived plasma,
measuring reflectance, transmission or transflectance of the test sample over a range of near-infrared wavelengths, thereby generating test wavelength spectra,
comparing the test wavelength spectra to a reference data set in the form of a model generated using multivariate analysis of processed reference wavelength spectra of reference samples having known concentrations of the analyte, to determine the concentration of the analyte in the sample.

In any aspect, the analyte is total protein or alcohol (e.g. ethanol). In these embodiments, the methods can then be used to determine the concentration of total protein or ethanol in a test sample obtained from processing of blood-derived plasma.

In any aspect, the preferred mode of measurement is transflectance.

In any embodiment, the light source in the near-infrared spectrum comprises a light source having a wavelength in the range of about 750 to about 2500 nm. In another embodiment, the light source comprises a wavelength from about 800 to 1100 nm. In yet another embodiment, the light source comprises a wavelength from about 1100 to about 2500 nm. Preferably the light source comprises a wavelength from about 1400 to about 2200 nm.

In a still further embodiment, the light source comprises a wavelength expressed in wavenumbers and the wavenumber is from about 4,000 to about 12,500 cm⁻¹.

In any embodiment, the wavelength spectra comprise measurements of reflectance, transmission, or transflectance at wavelengths in the range of about 750 to about 2500 nm. In another embodiment, the wavelength spectra comprise measurements at wavelengths from about 800 to 1100 nm. In yet another embodiment, the wavelength spectra comprise measurements of reflectance, transmission, or transflectance at wavelengths from about 1100 to about 2500 nm. Preferably the near infrared wavelength spectra comprise measurements at wavelengths from about 1400 to about 2200 nm.

In a still further embodiment, the wavelength spectra are expressed in wavenumbers and the wavenumber is from about 4,000 to about 12,500 cm⁻¹.

In any aspect, the model generation may include identification of signal changes in wavenumber regions of the spectra.

In one embodiment, particularly when the analyte is total protein, the wavenumber regions may include any one or more of about 9′000 to about 7′500 cm⁻¹, about 6′900 to about 5′600 cm⁻¹ and about 4′935 to about 4′500 cm⁻¹. In one embodiment, the wavenumber regions may include any one or more of 9′000 to 7′500 cm⁻¹, 6′900 to 5′600 cm⁻¹ and 4′935 to 4′500 cm⁻¹.

In one embodiment, particularly when the analyte is an alcohol such as ethanol, the wavenumber regions may include any one or more of 9′400-5′400 cm⁻¹, or 9′400-5′448 cm⁻¹.

In one embodiment, major water-derived signals are excluded. Typically, the major water-derived signals occur between 7′500 and 6′900 cm⁻¹ and between 5′600 and 4′935 cm⁻¹.

In one embodiment, particularly when the analyte is total protein and the sample obtained from processing of Cohn Fraction V (Fr V) or Kistler/Nitschmann Precipitate C, the wavenumber regions may include about 9′000 to about 7′500 cm⁻¹, and/or about 6′000 to about 5′600 cm⁻¹. In one embodiment, the wavenumber regions may include 9′000 to 7′500 cm⁻¹ and/or 6′000 to 5′600 cm⁻¹.

In any aspect, the model of the processed reference wavelength spectra is a model generated using partial least squares (PLS) regression of processed wavelength spectra of samples having known concentrations of the analyte is generated using a method described herein.

In another aspect, the present invention provides a method for generating a model to determine the concentration of an analyte in a sample obtained from plasma processing, the method comprising:
providing training samples obtained from processing of blood-derived plasma, wherein the samples have known concentrations of the analyte,
applying a light source in the near-infrared spectrum to the training samples,
measuring the reflectance, transmission or transflectance of the training samples over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentration of the analyte,
   thereby obtaining a model for determining the concentration of an analyte in a sample obtained from processing of blood-derived plasma. Optionally the multivariate analysis is selected from Partial least squares regression (PLS); PLS Discriminant Analysis (PLS-DA); Ordinary Least Squares (OLS) regression; MLR (multiple linear regression); OPLS (Orthogonal-PLS); SVM (support vector machines); GLD (general discriminant analysis); GLMC (generalized linear model); GLZ (generalized linear and non-linear model); LDA (Linear Discriminant Analysis); classification trees; cluster analysis; neural networks; and Pearson correlation.

In any aspect, the training samples are obtained from routine manufacture of blood-derived plasma products as further described herein and such as include immunoglobulins, and other proteins derived from blood plasma including albumin and clotting factors.

In any aspect, the spectral pre-treatment is 1^{st} derivative, vector normalization or a combination of both 1^{st} derivative, vector normalization. Alternatively, the spectral pre-treatment is min-max normalisation.

In any aspect, where the analyte is protein, the concentration of protein in reference or training samples may be determined using any means known in the art, for example the Dumas assay, or any means described herein.

In any aspect, where the analyte is an alcohol such as ethanol, the concentration of alcohol (e.g. ethanol) in the reference or training samples may be determined using any means known in the art, or using theoretical values, or any means described herein (e.g. gas chromatography or enzymatic ethanol determination).

In any aspect, the methods of the invention allow determination of protein concentration of a range of about 10 g/kg to about 150 g/kg, 10 g/kg to 150 g/kg, about 15 g/kg to about 45 g/kg, 15 g/kg to 45 g/kg, about 20 g/kg to about 35 g/kg, 20 g/kg to 35 g/kg, about 100 g/kg to about 150 g/kg or 100 g/kg to 150 g/kg. In one embodiment, where the protein in the test sample is predominantly, or contains a significant amount of, IgG the protein concentration range may be about 15 g/kg to about 40 g/kg, 15 g/kg or 40 g/kg, about 16 g/kg to about 42 g/kg, 16 g/kg to 42 g/kg, about 20 g/kg to about 35 g/kg, or 20 g/kg to 35 g/kg. In one embodiment, where the protein in the test sample is predominantly, or contains a significant amount of, albumin the protein concentration range may be about 100 g/kg to about 150 g/kg or 100 g/kg to 150 g/kg.

In any aspect, the methods of the invention allow determination of alcohol (e.g. ethanol) concentration of a range of about 1% v/v to about 65% v/v, or 1% v/v to 65% v/v, or about 8% to about 40% v/v, or 8% to 40% v/v.

In any aspect, the methods of the invention allow determination of total protein or ethanol concentration of a range typically used during the fractionation of blood plasma, including to produce any, or all of, Cohn Fraction I, Cohn Fraction (I+)II+III, Cohn Fraction IV (including Cohn Fraction IV₁, IV₄), and Cohn Fraction V and other similar variant fractions or precipitates. Further, in any aspect, the methods of the invention allow determination of total protein or ethanol concentration of a range typically used during the fractionation of blood plasma, including to produce any, or all of, Kistler/Nitschmann Precipitate A, Kistler/Nitschmann Precipitate B, Kistler/Nitschmann Fraction IV, and Kistler Nitschmann Precipitate C and other similar variant fractions or precipitates.

As used herein, Cohn Fraction (I+)II+III includes Cohn Fraction I+II+III or Cohn Fraction II+III. It is also equivalent to Kistler/Nitschmann Precipitate A and other similar variant fractions or precipitates.

As used herein, Cohn Fraction IV includes Cohn Fraction IV₁ and IV₄.

In any aspect, the methods of the invention allow determination of total protein or ethanol concentration of a range typically used during the fractionation of blood plasma to produce Cohn Fraction V. Further, in any aspect, the methods of the invention allow determination of ethanol concentration of a range typically used during the fractionation of blood plasma to produce either, or both of, Kistler/Nitschmann Precipitate C.

In any aspect, the methods of the invention allow determination of total protein or ethanol concentration of a range typically used during the dilution or resuspension of plasma fractions including any, or all of, Cohn Fraction I, Cohn Fraction (I+)II+III, Cohn Fraction IV (including Cohn Fraction IV₁, IV₄), and Cohn Fraction V and other similar variant fractions or precipitates. Further, in any aspect, the methods of the invention allow determination of total protein or ethanol concentration of a range typically used during the dilution or resuspension of plasma fractions including any, or all of, Kistler/Nitschmann Precipitate A, Kistler/Nitschmann Precipitate B, Kistler/Nitschmann Fraction IV, and Kistler Nitschmann Precipitate C and other similar variant fractions or precipitates.

In an embodiment, the total protein or ethanol concentration is measured during resuspension of any, or all of, Cohn Fraction I, Cohn Fraction II+III, Cohn Fraction I+II+III, or Kistler/Nitschmann Precipitate A or other similar variant fractions or precipitates.

In an embodiment, the total protein or ethanol concentration is measured during resuspension of Cohn Fraction IV paste (including Cohn Fraction IV₁, IV₄ or other similar variant fraction or precipitate).

In an embodiment, the total protein or ethanol concentration is measured after resuspension of any, or all of, Cohn Fraction I, Cohn Fraction II+III, Cohn Fraction I+II+III, or Kistler/Nitschmann Precipitate A paste and prior to any filtration (e.g. clarifying filtration) of the resuspended paste or any significant reduction in turbidity of the resuspended paste.

In an embodiment, the total protein or ethanol concentration is measured after resuspension of Cohn Fraction IV paste (including Cohn Fraction IV₁, IV₄ or other similar variant fraction or precipitate) and prior to any filtration (e.g. clarifying filtration) of the resuspended paste or any significant reduction in turbidity of the resuspended paste.

In an embodiment, Cohn Fraction I, Cohn Fraction (I+)II+III, Cohn Fraction IV paste (including Cohn Fraction IV₁, IV₄ or other similar fraction or precipitate), Kistler/Nitschmann Precipitate A, Kistler/Nitschmann Fraction IV or Kistler/Nitschmann Precipitate B, or other similar fraction or precipitate, paste is resuspended by the addition of one or more diluting agents, such as distilled water. Typically, the paste is resuspended by the addition of one or more diluting agents at a ratio of dilution agent between 1-7×the weight of the Precipitate paste. In an embodiment, the paste is resuspended at a temperature below 26° C., including 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., 0° C., −1° C., −2° C., −3° C., −4° C., −5° C., −6° C., −7° C. or −8° C. In an embodiment the resuspension temperature is <21° C.

In an embodiment, the ethanol concentration in the resuspended Cohn Fraction I, Cohn Fraction (I+)II+III, Cohn Fraction IV paste (including Cohn Fraction IV₁, IV₄ or other similar fraction or precipitate), Kistler/Nitschmann Precipitate A, Kistler/Nitschmann Fraction IV or Kistler/Nitschmann Precipitate B, or other similar fractions or precipitates, paste is between the range of about 2% (w/w) to about 30% (w/w), about 2% (w/w) to about 20% (w/w), about 5% (w/w) to about 30% (w/w), about 5% (w/w) to about 20% (w/w), about 5% (w/w) to about 15% (w/w), or about 5% (w/w) to about 10% (w/w).

In an embodiment, the protein concentration in the resuspended Cohn Fraction I, Cohn Fraction (I+)II+III, Cohn Fraction IV paste (including Cohn Fraction IV₁, IV₄ or other similar fraction or precipitate), Kistler/Nitschmann Precipitate A, Kistler/Nitschmann Fraction IV or Kistler/Nitschmann Precipitate B, or other similar fractions or precipitates, paste is between the range of about 5% (w/w) to about 15% (w/w), typically about 10% (w/w) to about 15% (w/w).

In an embodiment, optionally, filter aid is added to the resuspended Cohn Fraction I, Cohn Fraction (I+)II+III, Cohn Fraction IV paste (including Cohn Fraction IV₁, IV₄ or other similar fractions or precipitates), Kistler/Nitschmann Precipitate A, Kistler/Nitschmann Fraction IV or Kistler/Nitschmann Precipitate B, or other similar fractions or precipitates, paste prior to any filtration (e.g. clarifying filtration) step or prior to any significant reduction in turbidity of the resuspended paste.

In any aspect, the methods of the invention allow determination of total protein or ethanol concentration of a range typically used during the dilution or resuspension of Cohn Fraction V. Further, in any aspect, the methods of the invention allow determination of ethanol concentration of a range typically used during the dilution or resuspension of Kistler/Nitschmann Precipitate C.

In an embodiment, the total protein or ethanol concentration is measured during resuspension of Cohn Fraction V paste or Kistler/Nitschmann Precipitate C paste.

In an embodiment, the total protein or ethanol concentration is measured after resuspension of Cohn Fraction V paste or Kistler/Nitschmann Precipitate C paste and prior to any filtration (e.g. clarifying filtration) of the resuspended paste or any significant reduction in turbidity of the resuspended paste.

In an embodiment, Cohn Fraction V paste or Kistler/Nitschmann Precipitate C paste is resuspended by the addition of one or more diluting agents, such as distilled water. Typically, the Cohn Fraction V paste or Kistler/Nitschmann Precipitate C paste is resuspended by the addition of one or more diluting agents at a ratio of dilution agent between 1-3×the weight of the Precipitate paste. In an embodiment, the Cohn Fraction V paste or Kistler/Nitschmann Precipitate C paste is resuspended at a temperature below 26° C., preferably at or below 25° C., at or below 24° C., at or below 23° C., at or below 22° C., at or below 21° C., at or below 20° C., at or below 19° C., at or below 18° C., at or below 17° C., at or below 16° C., at or below 15° C., at or below 14° C., at or below 13° C., at or below 12° C., at or below 11° C., at or below 10° C., at or below 9° C., at or below 8° C., at or below 7° C., at or below 6° C., at or below 5° C., at or below 4° C., at or below 3° C., at or below 2° C., at or below 1° C. or 0° C. In an embodiment the resuspension temperature is <21° C.

In an embodiment, the ethanol concentration in the resuspended Cohn Fraction V paste or Kistler/Nitschmann Precipitate C paste is between the range of about 5% (w/w) to about 15% (w/w), typically about 5% (w/w) to about 10% (w/w).

In an embodiment, the protein concentration in the resuspended Cohn Fraction V paste or Kistler/Nitschmann Precipitate C paste is between the range of about 5% (w/w) to about 15% (w/w), typically about 10% (w/w) to about 15% (w/w).

In an embodiment, filter aid is added to the resuspended Cohn Fraction V paste or Kistler/Nitschmann Precipitate C paste prior to any filtration (e.g. clarifying filtration) step or prior to any significant reduction in turbidity of the resuspended paste.

In another aspect, the present invention provides a method for generating a model to determine the concentration of total protein or immunoglobulin G (IgG) during or after the resuspension of a Cohn Fraction I, Cohn Fraction II+III, Cohn Fraction I+II+III, Cohn Fraction IV paste, Kistler/Nitschmann Precipitate A or Kistler/Nitschmann Precipitate B paste, the method comprising:
resuspending Cohn Fraction I, Cohn Fraction II+III, Cohn Fraction I+II+III, Cohn Fraction IV paste, Kistler/Nitschmann Precipitate A or Kistler/Nitschmann Precipitate B paste with a suitable dilution agent to produce a series of training samples comprising different total protein or IgG concentrations, wherein the samples have known concentrations of total protein or IgG,
applying a light source in the near-infrared spectrum to the training samples,
measuring the reflectance, transmission or transflectance of the training samples over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentration of total protein or IgG.

In another aspect, the present invention provides a method for generating a model to determine the concentration of ethanol during or after the resuspension of a Cohn Fraction I, Cohn Fraction II+III, Cohn Fraction I+II+III, Cohn Fraction IV paste, Kistler/Nitschmann Precipitate A or Kistler/Nitschmann Precipitate B paste, the method comprising:
resuspending Cohn Fraction I, Cohn Fraction II+III, Cohn Fraction I+II+III, Cohn Fraction IV paste, Kistler/Nitschmann Precipitate A or Kistler/Nitschmann Precipitate B paste with a suitable dilution agent and stirring until the precipitate is dissolved,
optionally adding ethanol step wise to the resuspension and taking a training sample after each step wise addition to produce a series of training samples comprising different ethanol concentrations, wherein the samples have known concentrations of ethanol,
applying a light source in the near-infrared spectrum to the training samples,
measuring the reflectance, transmission or transflectance of the training samples over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentration of the ethanol.

In another aspect, the present invention provides a method for generating a model to determine the concentration of ethanol during or after the resuspension of a Cohn Fraction I, Cohn Fraction II+III, Cohn Fraction I+II+III, Cohn Fraction IV paste, Kistler/Nitschmann Precipitate A or Kistler/Nitschmann Precipitate B paste, the method comprising:
resuspending a Cohn Fraction I, Cohn Fraction II+III, Cohn Fraction I+II+III, Cohn Fraction IV paste, Kistler/Nitschmann Precipitate A or Kistler/Nitschmann Precipitate B paste with a suitable dilution agent and stirring until the precipitate is dissolved,
applying a light source in the near-infrared spectrum in the resuspension;
optionally adding ethanol step wise to the resuspension and measuring the reflectance, transmission or transflectance after each step of ethanol addition over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentrations of the ethanol.

In one aspect, the present invention provides a method for determining the concentration of total protein, IgG or ethanol during or after the resuspension of a Cohn Fraction I, Cohn Fraction II+III, Cohn Fraction I+II+III, Cohn Fraction IV paste, Kistler/Nitschmann Precipitate A or Kistler/Nitschmann Precipitate B paste for the purification of albumin, the method comprising:
applying a light source in the near-infrared spectrum to a test sample obtained at a time point during or at completion of the resuspension of the paste in a suitable dilution agent,
measuring reflectance, transmission or transflectance of the test sample over a range of near-infrared wavelengths, thereby generating test wavelength spectra,
comparing the test wavelength spectra to a reference data set in the form of a model generated using multivariate analysis of processed reference wavelength spectra of reference samples having known concentrations of total protein, albumin or ethanol, to determine the concentration of the total protein, albumin or ethanol in the sample.

In another aspect, the present invention provides a method for generating a model to determine the concentration of total protein or albumin during or after the resuspension of a Cohn Fraction V paste or Kistler/Nitschmann Precipitate C paste, the method comprising:
resuspending Cohn Fraction V paste or Kistler/Nitschmann Precipitate C paste with a suitable dilution agent to produce a series of training samples comprising different total protein or albumin concentrations, wherein the samples have known concentrations of total protein or albumin,
applying a light source in the near-infrared spectrum to the training samples,
measuring the reflectance, transmission or transflectance of the training samples over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentration of total protein or albumin.

In another aspect, the present invention provides a method for generating a model to determine the concentration of ethanol during or after the resuspension of a Cohn Fraction V paste or Kistler/Nitschmann Precipitate C paste, the method comprising:
resuspending Cohn Fraction V paste or Kistler/Nitschmann Precipitate C paste with a suitable dilution agent and stirring until the precipitate is dissolved,
optionally adding ethanol step wise to the resuspension and taking a training sample after each step wise addition to produce a series of training samples comprising different ethanol concentrations, wherein the samples have known concentrations of ethanol,
applying a light source in the near-infrared spectrum to the training samples,
measuring the reflectance, transmission or transflectance of the training samples over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentration of the ethanol.

In another aspect, the present invention provides a method for generating a model to determine the concentration of ethanol during or after the resuspension of a Cohn Fraction V paste or Kistler/Nitschmann Precipitate C paste, the method comprising:
resuspending Cohn Fraction V paste or Kistler/Nitschmann Precipitate C paste with a suitable dilution agent and stirring until the precipitate is dissolved,
applying a light source in the near-infrared spectrum in the resuspension;
optionally adding ethanol step wise to the resuspension and measuring the reflectance, transmission or transflectance after each step of ethanol addition over a range of near-infrared wavelengths, thereby generating training wavelength spectra,
selecting spectral regions of interest in the training wavelength spectra;
optionally applying at least one spectral pre-treatment;
generating a model by applying multivariate analysis to the spectra to provide a correlation with known concentrations of the ethanol.

In one aspect, the present invention provides a method for determining the concentration of total protein, albumin or ethanol during or after the resuspension of a Cohn Fraction V paste or Kistler/Nitschmann Precipitate C paste for the purification of albumin, the method comprising:
applying a light source in the near-infrared spectrum to a test sample obtained at a time point during or at completion of the resuspension of the paste in a suitable dilution agent,
measuring reflectance, transmission or transflectance of the test sample over a range of near-infrared wavelengths, thereby generating test wavelength spectra,
comparing the test wavelength spectra to a reference data set in the form of a model generated using multivariate analysis of processed reference wavelength spectra of reference samples having known concentrations of total protein, albumin or ethanol, to determine the concentration of the total protein, albumin or ethanol in the sample.

In any aspect, the training samples include concentrations of analyte across the concentration range for test sample determination. For example, if the possible concentration of an analyte in a test sample is within a range of X g/kg to Y g/kg, then the training samples include concentrations of analyte at, and between, X g/kg to Y g/kg.

In any aspect, the training samples and the test sample are exposed to a light source in the near-infrared spectrum at a temperature in the range of about −8° C. to about 37° C. or −8° C. to 37° C. Typically the temperature is in the range of about 10° C. to about 37° C., preferably in the range of about 15° C. to about 30° C. The temperature may be about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In any embodiment, the temperature is 18° C., 19° C., 20° C., 21° C., 22° C., 23° C., or 24° C.

In any aspect, the light source in the near-infrared range is applied to the training samples and/or the test sample using a probe adapted to emit light having wavelengths in the near-infrared range. Optionally the probe is configured for inclusion in an industrial protein mixing, filtration or purification apparatus, including for use for in-line measurement of reflectance, transmission or transflectance of the training samples over a range of near-infrared wavelengths.

In any aspect, the light source in the near-infrared range is applied to the training samples and/or the test sample during mixing of the samples. The light source may be applied to the sample(s) at an angle that is parallel to the direction of fluid stream during mixing of the sample(s). Alternatively, the light source may be applied to the sample(s) at an angle that is non-parallel to the direction of fluid stream during mixing of the sample(s), for example the light source may be applied to the sample(s) at, or about, 450 to the direction of the fluid stream during mixing of the sample(s).

In any aspect, the quality of the model generated may be judged using the following statistical parameters:
Number of latent variables (PLS factors) in the model,
Bias,
RMSECV,
RMSEP for independent test samples,
R², and/or
RPD value.

In any aspect, the sample comprising the analyte is obtained from processing of blood-derived plasma including any plasma sample derived from blood, preferably human blood. In certain embodiments, the sample is obtained or derived from the processing of blood-derived plasma that comprises fresh plasma, cryo-poor plasma, or cryo-rich plasma. In other words, the source of plasma may be blood, preferably human blood, preferably fresh plasma, cryo-poor plasma, or cryo-rich plasma. The plasma may be obtained from a number of donations and/or subjects, and pooled. The plasma may be hyperimmune plasma.

In any aspect, the sample comprising the analyte is a resuspension of a precipitate or paste obtained from blood-derived plasma and as further described herein.

In any aspect, the sample contains octanoic acid. Therefore, the octanoic acid containing sample also contains blood derived plasma or is obtained or derived from the processing of blood-derived plasma.

In any aspect of the present invention, the sample comprising the analyte is a blood-plasma fraction (intermediate). In particular embodiments the fraction is a Cohn Fraction. In a particularly preferred embodiment the plasma fraction is selected from the group consisting of Cohn Fraction I (Fr I), Cohn Fraction II+III (Fr II+III), Cohn Fraction I+II+III (Fr I+II+III), Cohn Fraction II (Fr II), Cohn Fraction III (Fr III), Cohn Fraction IV (Fr IV), Cohn Fraction V (Fr V), Kistler/Nitschmann Precipitate A, Kistler/Nitschmann Precipitate B, Kistler/Nitschmann Precipitate C. In another embodiment, the plasma fraction is selected from the group consisting of Cohn Fraction I (Fr I), Cohn Fraction II+III (Fr II+III), Cohn Fraction I+II+III (Fr I+II+III), or Kistler/Nitschmann Precipitate A (KN A, PPT A or Fr A). The plasma fraction may be a combination of different fractions. For example, the plasma fraction may be a combination of KN A and one or more of Fr I, Fr II+III and Fr I+II+III.

In any aspect, the sample comprising the analyte may comprise filter aid (for example, diatomaceous earth and perlite; or cellulose or silica gel).

In any aspect, the sample comprising the analyte is a turbid solution or suspension. In any embodiment, the turbid solution or suspension may have Nephelometric Turbidity Units (NTU) of equal to or greater than 10 NTU, equal to or greater than 15 NTU, equal to or greater than 20 NTU, equal to or greater than 25 NTU, equal to or greater than 30 NTU, equal to or greater than 35 NTU, equal to or greater than 40 NTU, equal to or greater than 45 NTU, equal to or greater than 50 NTU, equal to or greater than 55 NTU, equal to or greater than 60 NTU, equal to or greater than 65 NTU, equal to or greater than 70 NTU, equal to or greater than 75 NTU, equal to or greater than 80 NTU, equal to or greater than 85 NTU, equal to or greater than 90 NTU, equal to or greater than 95 NTU, equal to or greater than 100 NTU, equal to or greater than 150 NTU, equal to or greater than 200 NTU, equal to or greater than 250 NTU, equal to or greater than 300 NTU, equal to or greater than 350 NTU, equal to or greater than 400 NTU, equal to or greater than 450 NTU, equal to or greater than 500 NTU, equal to or greater than 550 NTU, equal to or greater than 600 NTU, equal to or greater than 650 NTU, equal to or greater than 700 NTU, equal to or greater than 750 NTU, equal to or greater than 800 NTU, equal to or greater than 850 NTU, equal to or greater than 900 NTU, equal to or greater than 950 NTU, equal to or greater than 1,000 NTU, equal to or greater than 1,500 NTU, equal to or greater than 2,000 NTU, equal to or greater than 2,500 NTU, equal to or greater than 3,000 NTU, equal to or greater than 3,500 NTU, equal to or greater than 4,000 NTU, equal to or greater than 4,500 NTU, equal to or greater than 5,000 NTU, equal to or greater than 5,500 NTU, equal to or greater than 6,000 NTU, equal to or greater than 6,500 NTU, equal to or greater than 7,000 NTU, equal to or greater than 7,500 NTU, equal to or greater than 8,000 NTU, equal to or greater than 8,500 NTU, equal to or greater than 9,000 NTU, equal to or greater than 9,500 NTU, or equal to or greater than 10,000 NTU. In any embodiment, the turbid solution or suspension may have NTU of 10 NTU to 100 NTU, 10 NTU to 90 NTU, 10 NTU to 80 NTU, 10 NTU to 70 NTU, 10 NTU to 60 NTU, 10 NTU to 50 NTU, 10 NTU to 40 NTU, 10 NTU to 30 NTU, 10 NTU to 20 NTU, 20 NTU to 100 NTU, 30 NTU to 100 NTU, 40 NTU to 100 NTU, 50 NTU to 100 NTU, 60 NTU to 100 NTU, 70 NTU to 100 NTU, 80 NTU to 100 NTU, or 90 NTU to 100 NTU. In any embodiment, the turbid solution may have a maximum NTU of 10,000 NTU, 9,500 NTU, 9,000 NTU, 8,500 NTU, 8,000 NTU, 7,500 NTU, 7,000 NTU, 6,500 NTU, 6,000 NTU, 5,500 NTU, 5,000 NTU, 4,500 NTU, 4,000 NTU, 3,500 NTU, 3,000 NTU, 2,500 NTU, 2,000 NTU, 1,500 NTU, 1000 NTU, 950 NTU, 900 NTU, 850 NTU, 800 NTU, 750 NTU, 700 NTU, 650 NTU, 600 NTU, 550 NTU, 500 NTU, 450 NTU, 400 NTU, 350 NTU, 300 NTU, 250 NTU, 200 NTU, 150 NTU, 100 NTU or 50 NTU.

In any aspect or embodiment, any or all steps of the method are performed in-line, at-line, off-line and/or on-line.

Those skilled in the field will understand and appreciate that plasma fractionation processes have some adaptability and have been optimized and varied over the years, for example, to suit different manufacturers and different product profile goals. An example of such a modification is the presence or absence of Cohn fractionation step IV-1, which can be used to extract alpha-1-antitrypsin. Thus, it should be understood that the methods and products described herein can be practiced with modifications and variations of human plasma fractionation processes, and that such modifications and variations are included within the scope of this disclosure.

In a preferred embodiment, the training samples comprise a representative set of samples that cover variables, such as different paste type, sample temperature, instrument variability, operator handling, raw materials, and plasma source.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Top: NIR raw spectra of one training set measured with transflectance (black) and reflectance (grey) probe. Bottom: Pre-treated (vector normalized+1st derivative) spectra.

FIG. 2a: Graphical summary of Mod_{gen}; protein concentration predicted by NIR (y-axis) vs. reference protein concentration (Dumas; x-axis) in g/kg. Each spectrum is represented by a single data point. Solid black line: predicted value=true value (slope=1). X-axis values b-r represent a range of 16 g/kg. Y-axis values b-k represent a range of 18 g/kg

FIG. 2b: Graphical summary of Mod_{lab}; protein concentration predicted by NIR (y-axis) vs. reference protein concentration (Dumas; x-axis) in g/kg. Each spectrum is represented by a single data point. Solid black line: predicted value=true value (slope=1). X-axis values b-o represent a range of 26 g/kg. Y-axis values a-g represent a range of 30 g/kg.

FIG. 3: Average protein concentrations of samples in test set predicted by Mod_{gen} [in g/kg]. X-axis values a-h represent a range of 14 g/kg. Y-axis values a-d represent a range of 15 g/kg.

FIG. 4: NIR spectra when resuspending Precipitate A (Fr A or PPT A) at 20° C. A. Raw spectra. B. 1^{st} derivative+vector normalisation pre-treated spectra. Grey: paste addition until temperature reaches 20° C.; black: resuspension. C. Predicted protein concentration [in g/kg] using NIR-based model compared to protein concentration determined according to Dumas between 2 and 24 hours following resuspension.

FIG. 5: NIR spectra when resuspending Cohn Fractions I+II+III (Fr I+II+III) at 20° C. A. Raw spectra. B. 1^{st} derivative+vector normalisation pre-treated spectra. Grey: paste addition until temperature reaches 20° C.; black: resuspension. C. Predicted protein concentration [in g/kg] using NIR-based model compared to protein concentration determined according to Dumas between 2 and 24 hours following resuspension.

FIG. 6: NIR spectra when resuspending Cohn Fractions II+III (Fr II+III) at 20° C. A. Raw spectra. B. 1^{st} derivative+vector normalisation pre-treated spectra. Grey: paste addition until temperature reaches 20° C.; black: resuspension. C. Predicted protein concentration [in g/kg] using NIR-based model compared to protein concentration determined according to Dumas between 2 and 24 hours following resuspension.

FIG. 7: 1^{st} derivative+vector normalisation pretreated spectra to Model_{comp} over manufacturing scale plasma fractionation of Cohn Fraction (I+)II+III and Cohn Fraction IV.

FIG. 8: Predicted ethanol concentration using NIR-based Model_{comp} compared to theoretical ethanol concentration [in % v/v]. Each spectrum is represented by a single data point. Solid black line: predicted value=true value (slope=1). X-axis values a-j represent a range of 45%. Y-axis values b-g represent a range of 50%.

FIG. 9: 1^{st} derivative+vector normalisation pretreated spectra to Model_{I+II+III} over manufacturing scale plasma fractionation of Cohn Fraction (I+)II+III.

FIG. 10: Predicted ethanol concentration using NIR-based Model_{I+II+III} compared to theoretical ethanol concentration [in % v/v]. Each spectrum is represented by a single data point. Solid black line: predicted value=true value (slope=1). X-axis values a-g represent a range of 30%. Y-axis values b-h represent a range of 30%.

FIG. 11: 1^{st} derivative+vector normalisation pretreated spectra to Model_{IV} over manufacturing scale plasma fractionation of Cohn Fraction IV.

FIG. 12: Predicted ethanol concentration using NIR-based Model_{IV} compared to theoretical ethanol concentration [in % v/v]. Each spectrum is represented by a single data point. Solid black line: predicted value=true value (slope=1). X-axis values a-j represent a range of 18%. Y-axis values a-j represent a range of 18%.

FIG. 13: NIR raw spectra of training set used for the Model_{Albresusp}.

FIG. 14: Graphical summary of Model_{Albresusp}; protein concentration predicted by NIR (y-axis) vs. reference protein concentration (Dumas; x-axis) in g/kg. Each spectrum is represented by a single data point. Solid black line: predicted value=true value (slope=1). X-axis values b-k represent a range of 45 g/kg. Y-axis values b-k represent a range of 45 g/kg

FIG. 15: Predicted protein concentration of independent test set using Model_{Albresusp} (y-axis) compared to reference protein concentration (Dumas; x-axis) in g/kg. Each spectrum is represented by a single data point. Solid black line: predicted value=true value (slope=1). X-axis values a-e represent a range of 20 g/kg. Y-axis values b-e represent a range of 15 g/kg.

FIG. 16: Pre-treated (1^{st} derivative) NIR raw spectra of training set used for the Model_{EtOH}.

FIG. 17: A) Graphical summary of Model_{EtOH}; ethanol concentration predicted by NIR (y-axis) vs. reference ethanol concentration (x-axis) in % w/w. Each spectrum is represented by a single data point. Solid black line: predicted value=true value (slope=1). X-axis values b-f represent a range of 4% w/w. Y-axis values b-f represent a range of 4% w/w. B) Predicted ethanol concentration of independent test set using Model_{EtOH} (y-axis) compared to reference ethanol concentration (x-axis) in % w/w. Each spectrum is represented by a single data point. Solid black line: predicted value=true value (slope=1). X-axis values b-h represent a range of 6% w/w. Y-axis values c-g represent a range of 4% w/w.

FIG. 18: Real-time ethanol concentration prediction during resuspension of Kistler-Nitschmann Precipitate C (squares, 2 runs) and Cohn Precipitate V (crosses) by in-line NIRS using Model_{EtOH}. Y-axis values a-d represent a range of 6% w/w.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

Due to the heterogeneity of plasma-derived product solutions and suspensions, the quantification of key chemical components, such as protein or alcohol (e.g. ethanol), is complex and to date, has only be achieved by use of off-line analytical methods. This can substantially impact on process efficiency.

The present invention seeks to address some of the deficiencies of prior approaches to processing of plasma-derived products by providing in-line systems for determining the concentration of various analytes in complex solutions during blood plasma processing. The methods of the present invention have the advantage of improving downstream efficiency, reduction in waste and/or improve final product yield.

The approach also enables quantification of analytes in various starting materials used during preparation of blood-plasma derived products without prior sample preparation, as is required with current in-line procedures. A further benefit of the methods of the present invention is the ability to monitor progression of product processing (such as resuspension) and other reactions in real-time leading to reduction of cycle time. The invention defined herein has been applied to manufacturing scale production to determine analyte concentration.

### Definitions

The term “a sample obtained from processing of blood-derived plasma” is intended to refer to any material, especially protein-containing material, derived from the fractionation or processing of blood plasma. The sample may be a suspension or concentrate or filtrate of a “protein-comprising precipitate”, wherein the “protein-comprising precipitate” is derived from blood plasma.

It will be appreciated that the samples (including the test sample) analysed in accordance with the methods described herein, do not need to be “isolated” samples. In other words, the term “sample” is intended to simply indicate a small part or quantity of a larger whole or bulk. The methods of the present invention are therefore intended to include at-line, in-line and off-line methods whereby the light source in the rear-infrared spectrum is applied to a small part of a larger bulk solution and where the light source can be applied to the small part of the bulk solution in situ, or to an aliquot of the solution that has been removed (isolated) from the larger bulk.

As used herein, the term “in-line” refers to a method of analysis whereby a probe, or sampling interface or sensor (eg for providing a light source in the near-infrared spectrum) can be placed directly in a process vessel or in line with a stream of flowing material to conduct the analysis. The process may involve placing a probe in a flow system or in a bioreactor. Such process may allow analysis without having to remove the probe or any material or samples from the bulk (ie the sample remains “in situ” for the analysis).

As used herein “on-line” refers to a method of analysis without having to remove the material or samples from the bulk. However, it may involve separating from the main process line and performing measurements on just a portion of the bulk. This may be accomplished by adding a sampling loop which directs a sample of the bulk material towards the probe or sensor, and whereby the diverted sample may be re-introduced to the process stream, flow or bulk of material, or disposed of, depending on the application.

As used herein, the term “at-line” refers to a method which includes manual sampling followed by discontinuous sample preparation, measurement and evaluation. When measuring at-line, analysis is typically completed at or near the process stream, flow or bulk of material.

As used herein, the term “off-line” refers to a method that involves the most physical difference between the process stream, flow or bulk of material and the analysis of the sample. Similarly to at-line measurement, off-line measurement involves removing an analytical sample from the larger bulk of material. Off-line analysis typically involves taking the sample or sometimes multiple samples to be analysed in a formal lab setting.

The term “protein-comprising precipitate” is intended to refer to any precipitated material containing a protein and derived from blood plasma. This term may refer to plasma, serum, precipitates produced from plasma or serum. Typically, in the context of the present invention, it refers to precipitates from plasma, such as Cohn or Oncley ethanol precipitates, or Kistler-Nitschmann precipitates.

As used herein, the coefficient of determination “R²” indicates the percentage of variance explained by the prediction model. The higher the coefficient, the better the correlation between the reference data and spectral data.

As used herein, “bias” is the Systematic averaged deviation between the reference values and the predicted values.

$$Bias = \frac{{\sum }_{i = 1}^{n} ( {y}_{C , i} - {Y}_{C , i} )}{n} \qquad \begin{matrix}{Y}_{C} = NIRS predicted value \\ {y}_{C} = Reference method value \\ n = Number of samples\end{matrix}$$

As used herein, for “cross validation” (also referred to as internal validation), individual leave-out samples (defined by the user) are removed from the calibration or training set. Using the remaining samples, a chemometric model is established and used to predict the previously extracted sample. A comparison of the predicted with the actual values determined by the reference method shows how well the model predicts the samples.

‘Partial Least Squares’ (Regression) is a statistical technique that reduces the predictors to a smaller set of uncorrelated components and performs least squares regression on these components, instead of on the original data.

As used herein ‘RMSECV’ is ‘root mean square error of cross validation’ and is a quantitative measure for the predictive ability of the model during cross validation. The RMSECV is comparable to the RMSEP for the external validation using an independent test set of samples.

$$RMSECV = \sqrt{\frac{{\sum }_{i = 1}^{n} {( {y}_{C , i} - {Y}_{C , i} )}^{2}}{n}} \qquad \begin{matrix}{Y}_{C} = NIRS predicted value of calibration set sample \\ {y}_{C} = Reference method value \\ n = Number of samples\end{matrix}$$

As used herein ‘RMSEP’ is the ‘root mean square error of prediction’ is a quantitative measure for the predictive ability of the model during external validation using an independent test set of samples. The RMSEP is comparable to the RMSECV for cross validation.

$$RMSEP = \sqrt{\frac{{\sum }_{i = 1}^{n} {( {y}_{T , i} - {Y}_{T , i} )}^{2}}{n}} \qquad \begin{matrix}{Y}_{T} = NIRS predicted value of independent test set sample \\ {y}_{T} = Reference method value \\ n = Number of samples\end{matrix}$$

As used herein, ‘RPD’ is Ratio of standard deviation (SD) and standard error of prediction (SEP).

$$RPD = \frac{SD}{SEP} \qquad \begin{matrix}SD = Standard deviation of reference values \\ SEP = Standard error of prediction\end{matrix}$$

Standard deviation may be determined by:

$$SD = \sqrt{\frac{1}{n - 1} {\sum }_{i = 1}^{n} {( {y}_{C , i} - \frac{{\sum }_{i = 1}^{n} {y}_{C , i}}{n} )}^{2}} \qquad \begin{matrix}{y}_{C} = Reference method value \\ n = Number of samples\end{matrix}$$

As used herein, ‘SEP’ is the ‘standard error of prediction’ is the RMSEP corrected by the bias.

$$SEP \sqrt{\frac{{\sum }_{i = 1}^{n} {( ( {y}_{T , i} - {Y}_{T , i} ) - Bias )}^{2}}{n - 1}} \qquad \begin{matrix}{Y}_{C} = NIRS predicted value of independent test set sample \\ {y}_{C} = Reference method value \\ n = Number of samples\end{matrix}$$

### Samples Comprising Analytes

The methods of the present invention relate to determining the amount or concentration of various analytes present in blood-plasma, fractions, or in derivatives or resuspensions of precipitated material derived therefrom. Typically, the analyte being determined comprises total protein, but may also comprise alternative components present in the samples, including alcohol (e.g., ethanol). The analyte may be an additive, i.e. an exogenous component added during the process, and is not naturally found in blood-plasma.

The plasma may be fresh plasma, “normal” plasma, “hyperimmune” plasma, cryo-poor plasma (also referred to as cryosupernatant), or cryo-rich plasma. Optionally, the plasma has been treated to remove components such as C1-inhibitor, PCC (Prothrombin Complex Concentrate) and/or AT-III. The plasma may be obtained from a number of donations and/or individuals, and pooled.

The term “cryosupernatant” (also called cryo-poor plasma, cryoprecipitate-depleted plasma and similar) refers to plasma (derived from either whole blood donations or plasmapheresis) from which the cryoprecipitate has been removed. Cryoprecipitation is the first step in most plasma protein fractionation methods in use today, for the large-scale production of plasma protein therapeutics. The method generally involves pooling frozen plasma that is thawed under controlled conditions (e.g. at or below 6° C.) and the precipitate is then collected by either filtration or centrifugation. The supernatant fraction, known to those skilled in the art as a “cryosupernatant”, is generally retained for use. The resulting cryo-poor plasma has reduced levels of Factor VIII (FVIII), von Willebrand factor (VWF), Factor XIII (FXIII), fibronectin and fibrinogen. Cryosupernatant provides a common feedstock used to manufacture a range of therapeutic proteins, including alpha 1-antitrypsin (AAT), apolipoprotein A-I (APO), antithrombin III (ATIII), prothrombin complex comprising the coagulation factors (II, VII, IX and X), albumin (ALB) and immunoglobulins such as immunoglobulin G (IgG).

The term “cryo-rich plasma” refers to plasma (derived from either whole blood donations or plasmapheresis) that has been frozen and then thawed, but from which the cryoprecipitate has not been removed.

Where plasma has been frozen for transport from a collection location, the frozen plasma is thawed and then collected in a pooling tank before centrifugation. The cryoprecipitate is removed by continuous centrifugation. The cryo-depleted plasma may be pumped into a stainless-steel fractionation tank and sampled for in-process controls

The plasma, whether pooled from more than one or several hundred individuals, or whether obtained from a single individual, may be hyperimmune plasma. For example, the plasma may be obtained from the blood of individual(s) who have/has mounted an immune response to an infection, and have recovered (and are therefore otherwise healthy individuals).

The sample comprising the analyte of interest may be a precipitate or fraction derived from processing of blood plasma. Many different methods can be used to selectively precipitate proteins from solution, for instance by the addition of salts, alcohols and/or polyethylene glycol with the combination of pH adjustment and/or a cooling step. It is therefore anticipated that the present invention will be applicable to most protein precipitates, such as immunoglobulin G-containing protein precipitates, regardless of how they are initially prepared. It should be noted that the present invention can also be implemented in separating other types of protein including albumin, immunoglobulins (Ig), such as IgA, IgD, IgE or IgM, either each type of immunoglobulin alone or a mixture thereof. It is foreseen that recombinant proteins are also suitable in this regard.

The sample may be any IgG or albumin-containing material (e.g. in form of a paste, precipitate, or inclusion bodies) or derived from a starting material such as a solution from which the IgG or albumin can be precipitated by for example one or more of the methods explained above, whether from plasma or serum of human or animal origin, fermentation broth, cell culture, protein suspension, milk or other original sources. The immunoglobulin-containing material or solution may contain monoclonal or polyclonal immunoglobulin(s). In some embodiments, the immunoglobulin-containing starting material is a solution comprising polyclonal antibodies. In other embodiments the starting material comprises a monoclonal antibody or a fragment thereof. In other embodiments, the sample may be any alcohol (e.g. ethanol) containing material or derived from a starting material such as a solution to which alcohol (e.g. ethanol) has been added to promote precipitation.

In order to obtain the immunoglobulins or albumin from plasma, the plasma is usually subjected to alcohol fractionation, which may be combined with other purification techniques like chromatography, adsorption or precipitation. However, other processes can also be used. For instance, the protein-comprising precipitate can be the II+III precipitate according to the Cohn's methods such as the Method 6, Cohn et. al. J. Am; Chem. Soc., 68 (3), 459-475 (1946), the Method 9, Oncley et al. J. Am; Chem. Soc., 71, 541-550 (1946), or the I+II+III precipitate, the Method 10, Cohn et. al. J. Am; Chem. Soc., 72, 465-474 (1950); as well as the Method of Deutsch et. al. J. Biol. Chem. 164, 109-118 (1946) or the Precipitate-A of Nitschmann and Kistler Vox Sang. 7, 414-424 (1962); Helv. Chim. Acta 37, 866-873 (1954). Alternative precipitates comprising the protein of interest include but are not limited to other immunoglobulin G or albumin-containing Oncley fractions, Cohn fractions, ammonium sulphate precipitates from plasma described by Schulze et al. in U.S. Pat. No. 3,301,842. Further alternative precipitates comprising the protein of interest include but are not limited to octanoic acid precipitates, as described, for example, in EP893450.

“Normal plasma”, “hyperimmune plasma” (such as hyperimmune anti-D, tetanus or hepatitis B plasma) or any plasma equivalent thereto can be used as a starting material in the cold ethanol fractionation processes described herein.

The supernatant of the 8% ethanol-precipitate (method of Cohn et al.; Schultze et al. (see above), p. 251), precipitate II+III (method of Oncley et al.; Schultze et al. (see above) p. 253) or precipitate B or IV (method of Kistler and Nitschmann; Schultze et al. (see Schultze above), p. 253) are examples of a source of IgG compatible with industrial scale plasma fractionation. The starting material for a purification process to gain IgG or albumin in high yield can alternatively be any other suitable material from different sources like fermentation and cell culture or other protein suspensions.

In the Cohn fractionation method, the first fractionation step results in fraction I which comprises mainly fibrinogen and fibronectin. The supernatant from this step is further processed to precipitate out fraction II+III and then fractions III and II. Typically, fraction II+III contains approximately 60% IgG, together with impurities such as fibrinogen, IgM, and IgA. Most of these impurities are then removed in fraction III, which is considered a waste fraction and is normally discarded. The supernatant is then treated to precipitate out the main IgG-containing fraction, fraction II, which can contain greater than 90% IgG. The above % values refer to % purity of the IgG. Purity can be measured by any method known in the art, such as gel electrophoresis or immune-nephelometry. In the Kistler & Nitschmann method, fraction I is equivalent to fraction I of the Cohn method. The next precipitate/fraction is referred to as precipitate A (fraction A). This precipitate is broadly equivalent, although not identical, to Cohn fraction II+III. The precipitate is then redissolved and conditions adjusted to precipitate out precipitate B (fraction B), which is equivalent to Cohn fraction III. Again, this is considered to be a waste fraction, and is normally discarded. The precipitate B supernatant is then processed further to produce precipitate II, which corresponds to Cohn Fraction II.

Particular protein-comprising precipitates or suspensions thereof can comprise plasma proteins, peptide hormones, growth factors, cytokines and polyclonal immunoglobulins proteins, plasma proteins selected from human and animal blood clotting factors including fibrinogen, prothrombin, thrombin, prothrombin complex, FX, FXa, FIX, FIXa, FVII, FVIIa, FXI, FXIa, FXII, FXIIa, FXIII and FXIIIa, von Willebrand factor, transport proteins including albumin, transferrin, ceruloplasmin, haptoglobin, hemoglobulin and hemopexin, protease inhibitors including β-antithrombin, α-antithrombin, α-2-macroglobulin, C1-inhibitor, tissue factor pathway inhibitor (TFPI), heparin cofactor II, protein C inhibitor (PAI-3), Protein C and Protein S, α-1 esterase inhibitor proteins, α-1 antitrypsin, antiangionetic proteins including latent-antithrombin, highly glycosylated proteins including α-1-acid glycoprotein, antichymotrypsin, inter-α-trypsin inhibitor, α-2-HS glycoprotein and C-reactive protein and other proteins including histidine-rich glycoprotein, mannan binding lectin, C4-binding protein, fibronectin, GC-globulin, plasminogen, blood factors such as erythropoietin, interferon, tumor factors, tPA, γCSF.

In certain embodiments, the methods of the present invention can be applied to determining the concentration of an analyte, eg total protein, during resuspension of a precipitate derived from blood-derived plasma. In particular, the methods can be used for assessing protein concentration in real-time during resuspension and to assist in determining total protein concentration to facilitate determination of the amount of subsequent reagents to the resuspension. The advantage of the methods of the invention is that the manufacturer does not need to manually sample the protein-containing sample to then manually calculate the amount of subsequent reagent to add. Moreover, the progression of protein dissolution during resuspension of the protein-containing precipitate or paste, such as described herein, can be monitored in real-time, enabling more efficient determination of when the resuspension is complete, optimum time for adding the subsequent reagents or performing the next step in product processing and thereby reducing unnecessary cycling time.

Much of the core methodology used to extract plasma proteins is largely based on the cryoprecipitation and ethanol fractionation. Albumin and IgG were the first proteins to have been fractionated from human plasma using multiple-step, sequential cold ethanol processes. Cohn and his colleagues were the pioneer in plasma fractionation by using low temperature and by the addition of ethanol from 8% to 40% v/v for separation of albumin. In Cohn's method, five fractions could be obtained which are from fraction I to fraction V, each of which is prepared by adjusting parameters such as the concentration of ethanol, concentration of protein, temperature, and pH.

In one example, the method is performed at large scale. For example, the method is performed on an industrial or a commercial scale. Methods of performing on an industrial or a commercial scale will be apparent to a skilled person and/or described herein. For example, the method performed on an industrial scale comprises large scale purification of IgG or albumin from plasma or fraction thereof.

In one example, large scale purification of IgG or albumin is performed using at least 500 kg of plasma or fraction thereof. For example, large scale purification of IgG or albumin is performed using between 500 kg to 1000 kg, or 1000 kg to 2500 kg, or 2500 kg to 5000 kg, or 5000 kg to 7500 kg, or 7500 kg, or 10000 kg, or 10000 kg to 12500 kg, or 12500 kg to 15000 kg of plasma or fraction thereof. In one example, large scale purification of IgG or albumin is performed using at least 1000 kg, or 2500 kg, or 5000 kg, or 7500 kg, or 10000 kg, or 12500 kg, or 15000 kg of plasma or fraction thereof. In one example, large scale purification of IgG or albumin is performed using at least 1000 kg of plasma or fraction thereof. In one example, large scale purification of IgG or albumin is performed using at least 2500 kg of plasma or fraction thereof. In one example, large scale purification of IgG or albumin is performed using at least 5000 kg of plasma or fraction thereof. In one example, large scale purification of IgG or albumin is performed using at least 7500 kg of plasma or fraction thereof. In one example, large scale purification of IgG or albumin is performed using at least 10000 kg of plasma or fraction thereof. In one example, large scale purification of IgG or albumin is performed using at least 12500 kg of plasma or fraction thereof. In one example, large scale purification of IgG or albumin is performed using at least 15000 kg of plasma or fraction thereof.

There are various stages during the processing of plasma into specific protein rich fractions that involve the use of ethanol and the present invention can be used to determine the amount of ethanol present in a complex solution which can then inform any adjustments that are required. Further, the present invention can be used to determine when a certain concentration of ethanol has been reached during a step of ethanol addition.

As described herein, the sample comprising the analyte may be a turbid solution or suspension, particularly a highly turbid solution or suspension. In any embodiment, (a) the turbid solution or suspension may have NTU of equal to or greater than any value described herein, (b) the turbid solution or suspension may have NTU of any value described herein, or (c) the turbid solution or suspension may have a maximum NTU of any value described herein. Typically turbidity is measured using various methods of photometry of turbid media, such as nephelometry, optometry, turbidimetry. Turbidity measurements are made using an instrument such as a turbidity meter or nephelometer. Typically, this is a photoelectric detector that measures the light scattered by a liquid. In particular, it is the scattering of light by suspensions that makes it possible to estimate the concentration of substances suspended in a liquid. Usually this device consists of a white light or infrared light source. In nephelometry, scattered light is measured at 90° and 25° with respect to the incident light. In turbidimetry, scattered light is measured using a sensor located on the axis of the incident light. Such turbidity analysis methods are well known in the art and a wide range of instruments are available for turbidity analysis, including hand-held and in-line sensors, for example the Hach TL2360 hand-held turbidimeter that measures turbidity in nephelometric turbidity units (NTU) at a 90° angle. In any method of the invention described herein, the method further provides a step of determining the turbidity of a sample obtained from processing of blood-derived plasma. Preferably, the turbidity of the sample is any value or range described herein. Preferably, the step of determining the turbidity of a sample comprises measuring turbidity in a 10 mL volume of a test sample obtained from processing of blood derived plasma in 11 mm glass tubes using a Hach TL2360 turbidimeter calibrated with NTU primary Formazin solution standards, at a 90° angle.

### Methods for Obtaining Wavelength Spectra

The skilled person will be familiar with standard equipment that can be used for applying light sources in the near-infrared range. In the context of the present invention, and in the preferred embodiments relating to determining protein concentration during processing of blood-derived plasma samples, the equipment may include use of an NIR probe adapted for use in a large vessel which comprises the samples of interest.

In one embodiment the NIR spectroscopy instrument is arranged to analyze the test sample during mixing in a large tank and provide NIR data in real-time. In certain embodiments, several probes may be connected to a single spectrometer. In an embodiment, a first probe may thus be arranged at the first position while a second probe is arranged at the second position and, if applicable, a third probe is arranged at the third position. All such probes may be connected to the same spectrometer. The skilled person will appreciate that the use of multiple NIR probes may assist with providing a more accurate range of data relating to test samples or training samples comprising the analyte of interest.

The probe of the NIR spectroscopy instrument may be in the form of an immersion probe or constitute a part of a flow cell. The whole process flow or a side stream of the flow can be lead through such a flow cell.

In preferred embodiments, the NIR probe is configured to enable measurement of NIR spectra during mixing of a sample. The optical slit of the NIR probe may be oriented parallel to the direction of the fluid stream during mixing. Typically, the optical slit of the NIR probe is oriented so that it is not directly facing the flow of the fluid stream during mixing. For example, the optical slit may be perpendicular or at an angle relative to the fluid stream during mixing. In other words, the NIR probe may be oriented downwards alongside the wall of the vessel.

### Methods for Generating Models/Reference Data Sets

The skilled person will be familiar with general approaches for preparing a reference data set or model of representative NIR spectra against which the spectra from test samples can be compared for the purposes of determining analyte concentration.

The reference data set may be from one or more samples comprising a known concentration of the analyte, wherein the concentration of the analyte has been determined by a method that is appropriate given the composition of the reference and test samples. For example, in the context of turbid solutions or suspensions comprising proteins, the most appropriate method for confirming protein concentration may be the Dumas method which is based on determining total nitrogen content, rather than other methods for determining protein concentration, such as the Biuret assay, BCA assay, Bradford assay or absorbance at 280 nm.

Representative NIR spectra can then be obtained for the reference or training samples for which protein concentration has been determined, such that the representative NIR spectra can be used to form the basis of a model against which test wavelength spectra can be assessed.

The skilled person will appreciate that the greater the number of representative NIR spectra or training spectra provided, the greater the accuracy of the model.

There may be a need to apply spectral pre-treatments to data (whether the test spectra or the reference or training spectra used to derive a suitable model). These pre-treatments can be applied to emphasise spectral changes. Examples of suitable spectral pre-treatments include vector normalisation, first derivative, min-max normalisation, straight line subtraction, multiplicative scatter correction, 2^{nd} derivative and combinations thereof. Preferably, the pre-treatment applied to the test spectra or the reference or training spectra used to derive a suitable model is vector normalisation or 1^{st} derivative. In one embodiment, the pre-treatment applied to the test spectra or the reference or training spectra used to derive a suitable model is vector normalisation in combination with 1^{st} derivative.

The model may be generated using a multivariate calibration algorithm, such as Multiple Linear Regression (MLR), Principal Component Regression (PCR), or Partial Least Squares (PLS)-Regression. Preferably the model is generated using Partial Least Squares (PLS)-Regression, such as that described herein. The PLS algorithm is described in (Haaland, Thomas, Anal. Chem 60 (1998) 1193; Martens, Naes, Multivariate Calibration, J. Wiley & Sons, New York (189): Chapter 3.5; Brown, Apply. Spectosc. 49, No. 12 (1995) 14A; and Bouveresse, Hartmann, Massard, Last, Prebble, Anal. Chem. 68, No. 6 (1996) 982).

Methods for assessing the quality of a given model (including to then determine whether further training data are required for further developing the model) are described herein.

In certain examples, criteria that may be considered when assessing the model quality of the different chemometric models or multivariate models include:
Rank: corresponds to the number of factors of the chemometric model. A lower rank usually leads to increased model stability.
Root mean square error of cross validation (RMSECV): The RMSECV should be minimized.
Residual prediction deviation (RPD): model performance indicator. The RPD should be maximized.
R²: coefficient of determination, describes the relation between spectral data and the concentration data. The R² should be maximized to close to 100.

The following criteria may also be considered when assessing the predictive ability of the chemometric models or multivariate models on an independent data set:
Bias: Average difference between reference values and predicted values. Should be close to 0.
Root mean square error of prediction (RMSEP): accuracy indicator for prediction of independent test samples. The RMSEP should be minimized.
Residual prediction deviation (RPD): model performance indicator. The RPD should be maximized.
R²: coefficient of determination, describes the relation between spectral data and the concentration data. The R² should be maximized to close to 100.

The generation of the model may involve training samples that may comprise a representative set of samples that cover variables, such as different paste type, sample temperature, instrument variability, operator handling, raw materials, and plasma source. Using such varied reference samples to capture such variables in the generation of the training model will further ensure the robustness of the model when it comes to assessing a variety of samples comprising analytes of unknown concentration.

### EXAMPLES

### Example 1—Description of NIR Measurement Setup for at-Line Protein Determination in Ig Precipitate Suspension

### NIR Spectrometer

The NIR measurements were conducted with a FT-NIR Matrix-F process spectrometer from Bruker Optics GmbH. The NIR process spectrometer can be used for spectroscopic analysis of liquids, suspensions and solids by transmission, diffuse reflectance and transflectance (the latter two are used here).

### NIR Probes

NIR spectra of the training samples were mainly acquired with a transflectance probe (details see Table 1). Few spectra were also recorded with a reflectance probe in order to compare these two measurement principles and select the most appropriate for the intended purpose

TABLE 1
NIR probes used in the feasibility study.

| | Measurement | Slit width/Optical path | Optical fiber |
|---|---|---|---|
| NIR probe | principle | length (OPL) | length |
| IN271 (Bruker) | Transflectance | Slit: 1 mm/OPL: 2 mm | 5 m |
| Reflector-NIR- | Reflectance | n/a | 5 m |
| 12S-300H | | | |
| (Solvias) | | | |

### Software

Spectra acquisition and data manipulation was conducted with the following software programmes:
Bruker OPUS: Basic software for operating the spectrometer, spectra acquisition and definition of the instrument parameters.
Bruker OPUS QUANT: Software package used for generation, optimization and validation of NIR model as well as for quantitative analysis.

### At-Line Measurement Setup

The measurement setup for NIR at-line data acquisition consisted of the following components a magnetic stirrer, a laboratory lifter, a magnetic stir bar (2.5 cm length), a stand and a clamp.

NIR spectra were recorded with 64 scans in the spectral range between 4000⁻¹ and 11000 cm⁻¹ at a resolution of 16 cm⁻¹.

A background spectrum of air was recorded and used to correct the sample NIR spectra. The spectra of all samples were recorded in triplicate.

In the case of obvious spectral outliers (as recognized by shape of the spectrum, e.g. due to air entrapped in slit) all replicate spectra of the respective sample were excluded manually from the training and test data sets.

The NIR probe was fixed with a stand and clamp. A magnetic stir bar was added to the sample container and the sample was stirred for 2 min at 450 rpm. Then, the sample was elevated on the laboratory lifter until the probe was immersed in the sample. The sample was stirred for an additional minute to ensure a homogeneous distribution of the suspension in the slit of the transflectance probe.

### Analytical Reference Method—Dumas Assay

The concentration of total protein in training samples was quantified by the Dumas method for total nitrogen determination (European Pharmacopoeia, Chapter 2.5.33 “Total Protein”, Method 7, Procedure B, current version; US Pharmacopoeia <1057>“Biotechnology-Derived Articles—Total Protein Assay”, Method 7, Procedure 2, current version; JP Pharmacopoeia, G3 “Biotechnological/Biological Products—Total Protein Assay”, Method 7, Procedure B, current version). The result is reported in g/kg. All reference samples were stored at 2° C.-8° C. prior to analysis.

### Sample Preparation

### Laboratory Samples

In the laboratory, samples of resuspended IgG precipitate from different precipitate types and different precipitate lots were prepared with sodium acetate buffer in 100 ml vacuum flasks. The total sample volume was approx. 70 mL.

All laboratory samples were measured in triplicate at room temperature of approx. 21° C. Additionally, some samples were also measured at approx. 15° C. and approx. 30° C. to 35° C.; for temperature adjustment, the samples were tempered in a water bath prior to the NIR measurement. If the samples were not measured on the same day as they were prepared, they were stored at 4° C. until the measurement.

### Routine Samples

For each routine lot two independent samples were transferred in 100 ml vacuum flasks in the Ig manufacturing facilities. NIR spectra of the samples were recorded in the laboratory and/or on NIR spectrometers available in the routine manufacturing facilities.

Routine samples were measured in triplicate at room temperature of approx. 21° C. Additionally, some samples were also measured at approx. 15° C. and approx. 30° C. to 35° C.; for temperature adjustment, the samples were tempered in a water bath prior to the NIR measurement. If the samples were not measured on the same day as they were prepared, they were stored at 4° C. until the measurement.

### Chemometric Models or Multivariate Models

### Generation of Models Using QUANT

In a typical NIR spectrum of a resuspended IgG precipitate sample there are two major peaks at approx. 6,900 cm⁻¹ and 5,000 cm⁻¹ wavenumber that reflect total absorption of the incoming light by water contained in the matrix. As hardly any light reaches the detector in these spectral regions, no quantitative information can be reliably extracted. For this reason, the spectral regions around these peaks were not included in the models.

In order to emphasize spectral changes, raw spectra are often pre-treated. The pre-treated spectra are the basis for the generation of chemometric quantitative models. The applied spectra pre-treatments were 1^{st} derivative, vector normalization and the combination of 1^{st} derivative and vector normalization.

All models were generated based on the acquired spectra and the corresponding protein reference values using the Bruker QUANT software package. The optimum combination of the spectral ranges and the pre-treatment of the spectra was determined in the software's optimization tool. Cross validation was used for internal validation of the models.

### Model Quality Criteria

The following criteria were considered when assessing the model quality of the different chemometric models or multivariate models:
Rank: corresponds to the number of factors of the chemometric model. A lower rank usually leads to increased model stability.
Root mean square error of cross validation (RMSECV): The RMSECV should be minimized.
Residual prediction deviation (RPD): model performance indicator. The RPD should be maximized.
R²: coefficient of determination, describes the relation between spectral data and the concentration data. The R² should be maximized to close to 100.

The following criteria were considered when assessing the predictive ability of the chemometric models or multivariate models on an independent data set:
Bias: Average difference between reference values and predicted values. Should be close to 0.
Root mean square error of prediction (RMSEP): accuracy indicator for prediction of independent test samples. The RMSEP should be minimized.
Residual prediction deviation (RPD): model performance indicator. The RPD should be maximized.
R²: coefficient of determination, describes the relation between spectral data and the concentration data. The R² should be maximized to close to 100.

### Example 2—Probe Selection for at-Line Protein Determination in Ig Precipitate Suspension: Transflectance Mode Vs Reflectance Mode

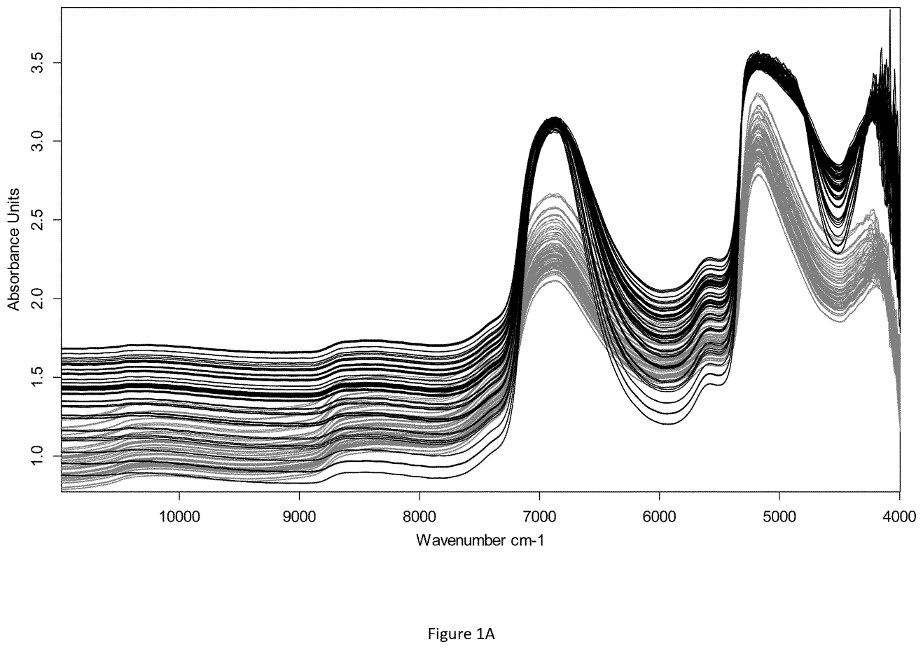

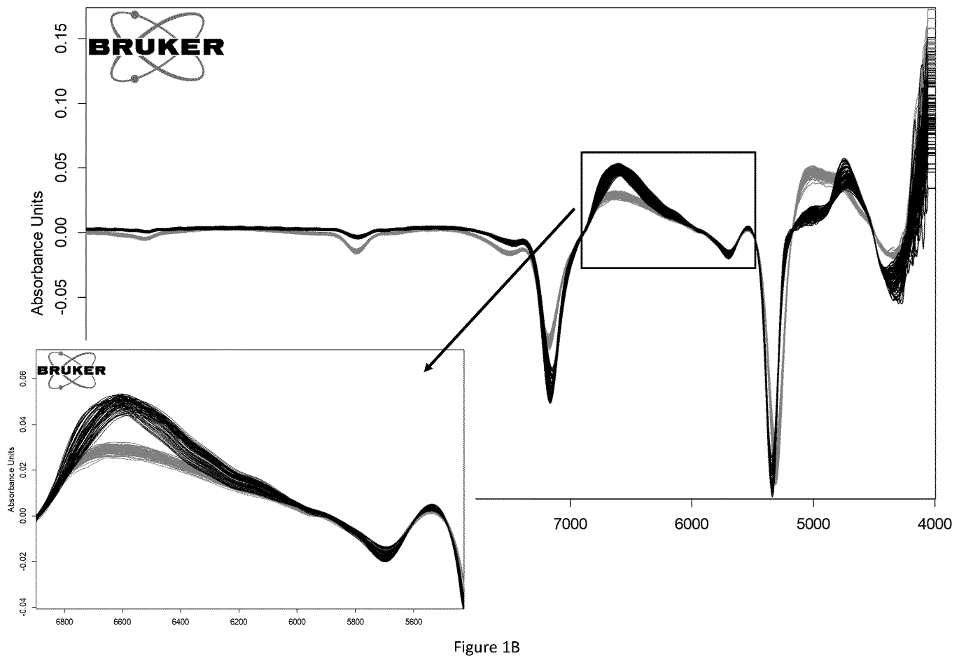

A series of training samples for one type of Ig precipitate suspension was measured with both transflectance and reflectance probe. The spectra of samples obtained with the two probe types are depicted in FIG. 1. Based on the spectra of the training samples and the corresponding protein reference values a separate model for each measuring mode was generated. The spectral ranges during the optimization were set to five default ranges (9400-7500 cm-1, 7500-6100 cm-1, 6100-5450 cm-1, 5450-4600 cm-1, 4600-4250 cm-1). The properties of the NIR models are listed in Table 2.

TABLE 2
Model properties for transflectance and reflectance data

| Model properties | Transflectance mode | Reflectance mode |
|---|---|---|
| RMSECV | 0.374 | 0.546 |
| Rank | 5 | 6 |
| RPD | 21.1 | 14.4 |
| R² | 99.77 | 99.52 |

From both sets of spectral data it was possible to generate a model of high quality. The model generated from the transflectance data resulted in a lower error (RMSECV), lower rank, higher RPD and R² compared to the model generated from the reflectance data. Therefore, the transflectance probe was preferred to the reflectance probe. All further NIR studies for Ig precipitate suspension were continued with the NIR transflectance probe.

### Example 3—at-Line Protein Determination in Ig Precipitate Suspension

### General NIR Models

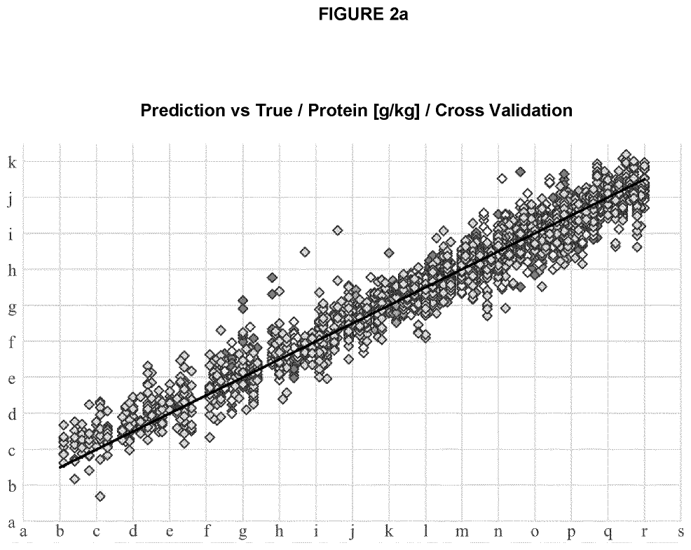

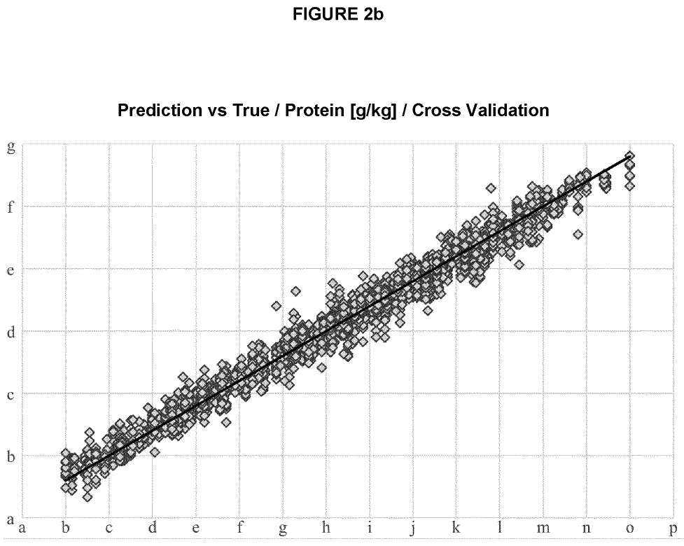

The NIR models Mod_{gen} (FIG. 2a) and Mod_{lab} (FIG. 2b) were developed to facilitate the prediction of the protein concentration in Ig precipitate suspension of different precipitate types (PPT A, Fr I+II+III, Fr II+III).

Multivariate NIR models were generated by partial least squares (PLS) regression using the OPUS QUANT software package to process raw spectra of training samples in conjunction with the respective protein references values (Dumas).

Based on signal changes in the NIR loading plots the wavelength regions of approx. 9′000-7′500 cm⁻¹, 6′900-5′600 cm⁻¹ and 4′935-4′500 cm⁻¹ were selected for NIR model optimization; the major water-derived signals between 7′500 and 6′900 cm⁻¹ and between 5′600 and 4′935 cm⁻¹ were excluded.

NIR models were optimized with a pre-defined selection of spectral pre-treatments, i.e. 1^{st} derivative, vector normalization, and the combination of 1^{st} derivative and vector normalization.

A preliminary set of NIR models was chosen and validated by internal cross validation with a standard number of 1 leave-out sample per 30 samples contained in the model. The model of choice was selected from the preliminary set of NIR models by thorough assessment of the statistical quality attributes of the internal validation results and the quality of prediction for independent test samples.

### Model Robustness Testing

In order to ensure that the model comprises a representative set of spectra, model generalization (i.e. the model's ability to adapt properly to new, previously unseen data, drawn from the same distribution as the one used to create the model) is continuously assessed during data collection. Testing the ability of the model to generalize is referred to as “model robustness testing”.

In short, the RMSECV and R² values obtained from cross validation of the model are compared to:
1. The RMSEP and R² values obtained from test set validation of the same data set randomly divided into two equal subsets (one training set and one test set) using the same spectral regions and pre-treatments as for the initially performed cross validation.
2. The RMSEP and R² values obtained from test set validation of the same data set randomly divided into two equal subsets (one training set and one test set, interchanged from 1) using the same spectral regions and pre-treatments as for the initially performed cross validation.

If no or little difference in the error tolerances (RMSECV/RMSEP and R²) is observed between cross validation and the two test set validations the model is considered sufficiently stable. Otherwise, the model should be further improved (e.g. by augmenting with additional spectra), in order to enhance the predictive ability of the model.

Properties of model Mod_{gen}

Table 3 provides a summary of the optimized NIR model Mod_{gen}; a visualization of Mod_{gen} is presented in FIG. 2a.

TABLE 3
Properties and quality attributes of Mod_{gen}
Property/quality attribute

| | | |
|---|---|---|
| No. of samples | 1′012 samples | Laboratory (dilution series)*: |
| | | n = 653 |
| | | PPT A: n = 228 |
| | | Fr I + II + III: n = 231 |
| | | Fr II + III: n = 194 |
| | | Laboratory (routine samples)*: |
| | | n = 210 |
| | | PPT A: n = 84 |
| | | Fr I + II + III: n = 84 |
| | | Fr II + III: n = 42 |
| | | Large scale facilities (routine |
| | | samples): n = 149 |
| | | PPT A: n = 62 |
| | | Fr I + II + III: n = 75 |
| | | Fr II + III: n = 12 |

| Spectral pre-treatments | Vector normalization |
|---|---|
| Calibration regions | 6′773 - 5′593 cm⁻¹, 4′775 - 4′497 cm⁻¹ |
| No. of PLS factors | 15 |
| (rank) | |
| RMSECV [g/kg] | 0.860 |
| R² | 94.73 |
| RPD | 4.35 |
| Bias | −0.00112 |
| Offset | 1.57 |
| Correlation | 0.9733 |
| coefficient R | |
| Slope | 0.947 |

| Model robustness | | Comment |
|---|---|---|
| Test | RMSEP = 0.843 | The model is considered robust because similar |
| set A | R² = 94.68 | values were obtained for RMSEP of the |
| Test | RMSEP = 0.853 | individual test sets and RMSECV of the |
| set B | R² = 95.05 | training set. Further, no increase in error was |
| | | observed with smaller sample size in the |
| | | training set. |

*Each sample was measured in the laboratory at three different temperatures.

### Properties of Model Mod_{Lab}

Table 4 provides a summary of the optimized NIR model Mod_{lab}; a visualisation of Mod_{lab} is presented in FIG. 2b across a protein range from 16 to 42 g/kg.

TABLE 4
Properties and quality attributes of Mod_{lab}
Property/quality attribute

| | | |
|---|---|---|
| No. of samples | 1′041 samples | Laboratory (dilution series)*: |
| | | n = 653 |
| | | PPT A: n = 258 |
| | | Fr I + II + III: n = 300 |
| | | Fr II + III: n = 269 |

| | |
|---|---|
| Spectral pre-treatments | First derivative |
| Calibration regions | 9′003 - 7′498 cm⁻¹, 6′403 - 5′593 cm⁻¹, |
| | 4′605 - 4′497 cm⁻¹ |
| No. of PLS factors (rank) | 14 |
| RMSECV [g/kg] | 0.843 |
| R² | 97.7 |
| RPD | 6.59 |
| Bias | −0.0136 |
| Offset | 0.711 |
| Correlation coefficient R | 0.9884 |
| Slope | 0.976 |

### Predictive Ability of Mod_{Gen}

The predictive ability of the NIR model Mod_{gen} was assessed with a set of independent test samples (i.e. samples not contained in the models) from Ig routine manufacture covering suspensions of the different Ig precipitate types (PPT A, Fr I+II+III, Fr II+III). NIR spectra of the routine test samples (2 independent samples, each measured in triplicate) were acquired on different spectrometers.

If the absolute difference between the mean of the 2×3 routine spectral replicates exceeded a maximum value of 1.5 g/kg all replicate spectra of the respective sample were removed from the test data set.

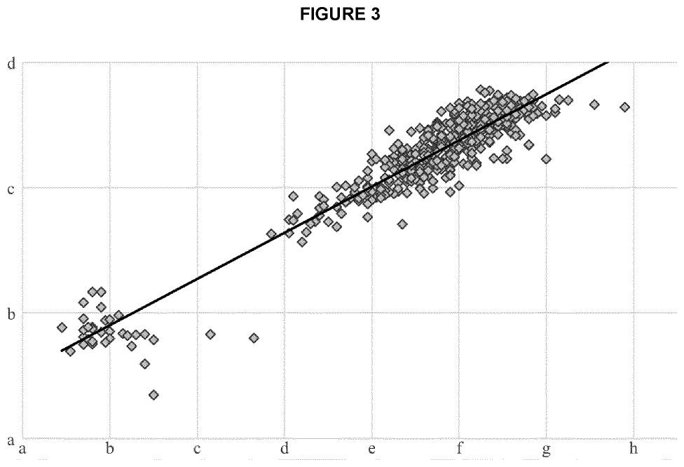

A summary of the prediction across the test data set covering the different precipitate types is provided in Table 5 and FIG. 3.

The RMSEP for the entire test set containing the different precipitate types was 0.880 g/kg and thus very similar to the RMSECV of 0.860 g/kg for Mod_{gen} (Table 5), indicating robustness of the prediction independent samples by Mod_{gen}. Furthermore, the prediction of samples in the test set is characterized by a negligible bias of −0.0624, an offset of 2.446, a slope of 0.920 and a correlation coefficient (R) of 0.9277.

TABLE 5
Summary of statistical quality attributes and test set composition
used to determine the predictive ability of Mod_{gen} across the entire
set of independent samples covering different precipitate types.

| Property/quality attribute | | Comment |
|---|---|---|
| Total no. of | 467 samples | Large scale facilities (routine samples): |
| samples in test set | | PPT A: n = 320 |
| | | Fr I + II + III: n = 114 |
| | | Fr II + III: n = 33 |

| | |
|---|---|
| RMSEP [g/kg] | 0.880 |
| RPD | 2.64 |
| Bias | 0.0624 |
| Offset | 2.446 |
| Correlation | 0.9277 |
| coefficient (R) | |
| Slope | 0.920 |

### Example 4—in-Line Protein Determination in Ig Precipitate Suspension

400 g to 900 g of Ig precipitate (PPT A, Fr I+II+III, Fr II+III) was resuspended in a scale-down double-jacketed steel reactor under tight temperature control. NIR process monitoring of Ig precipitate resuspension was started after the suspension reached a target temperature of 20° C. and continued for approx. 24 hours.

The NIR setup comprised a Matrix FT-NIR process spectrometer with a IN271 transflectance probe and 5 m optical fiber. The NIR probe was introduced into the level through an under-level port and configured to enable measurement of NIR spectra during mixing of a sample. The optical slit of the NIR probe was oriented parallel to the direction of the fluid stream during mixing.

The conditions for NIR data acquisition were equivalent to the at-line NIR system described in Example 1. NIR spectra were recorded at intervals of 3 minutes.

NIR models developed with at-line samples of Ig precipitate suspension were used for in-line protein quantification during the Ig precipitate resuspension reaction.

Additionally, reference samples were taken after 2 h and approx. 24 h and analysed with the Dumas protein assay.

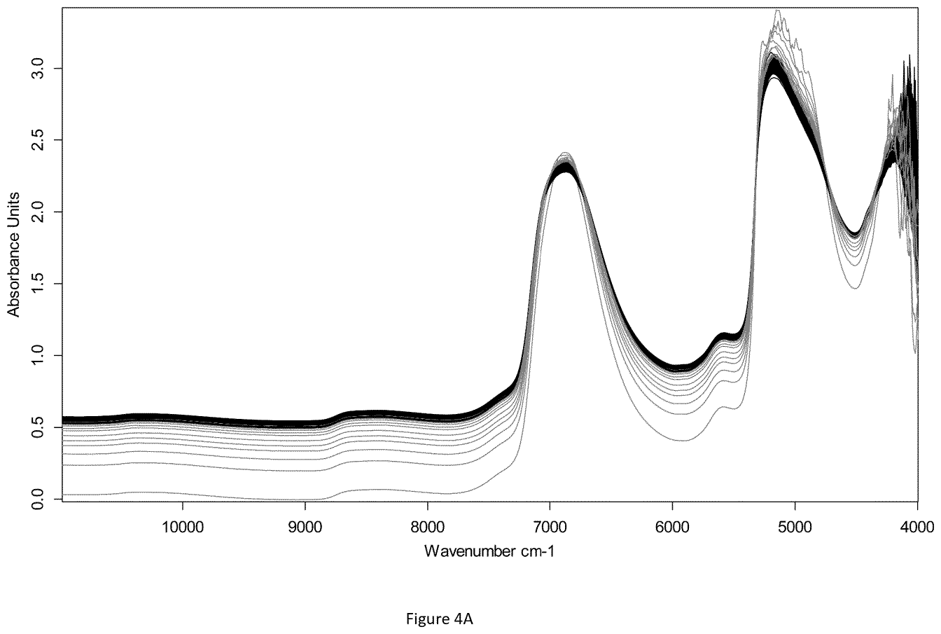

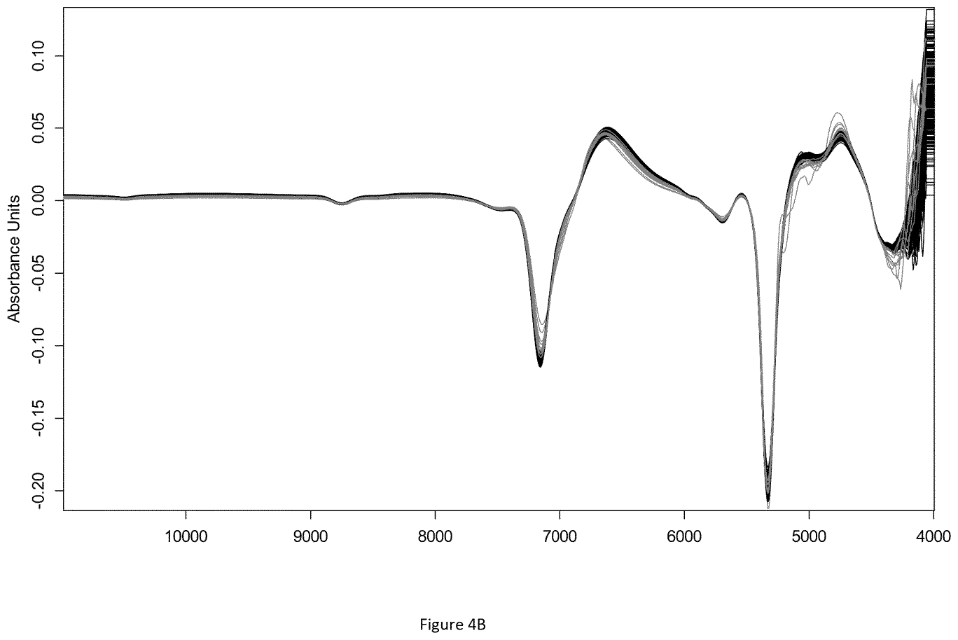

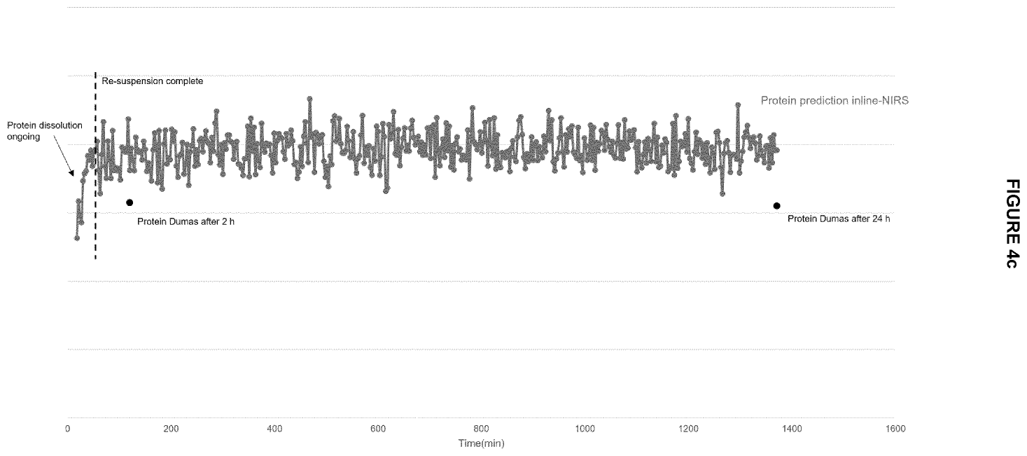

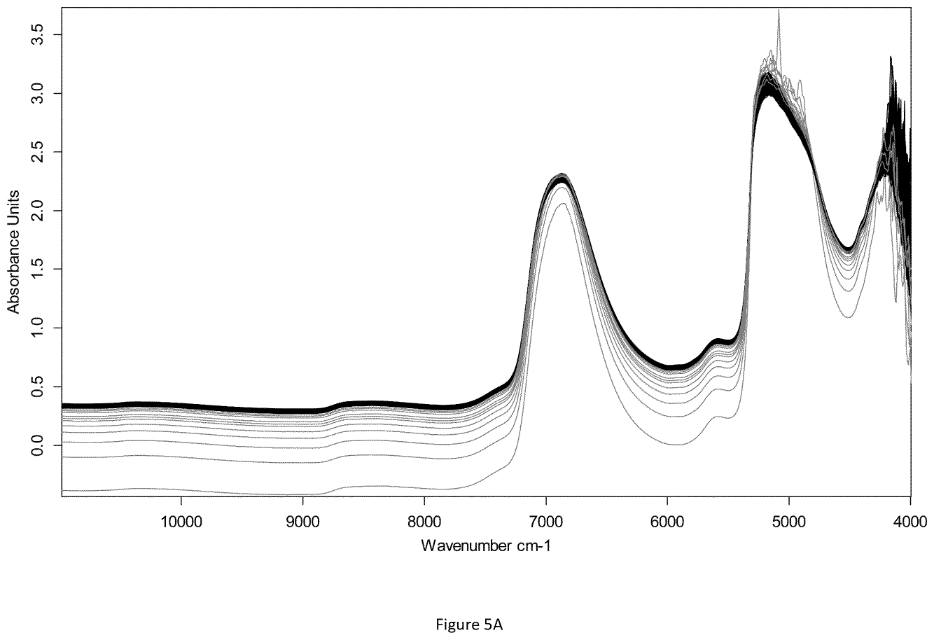

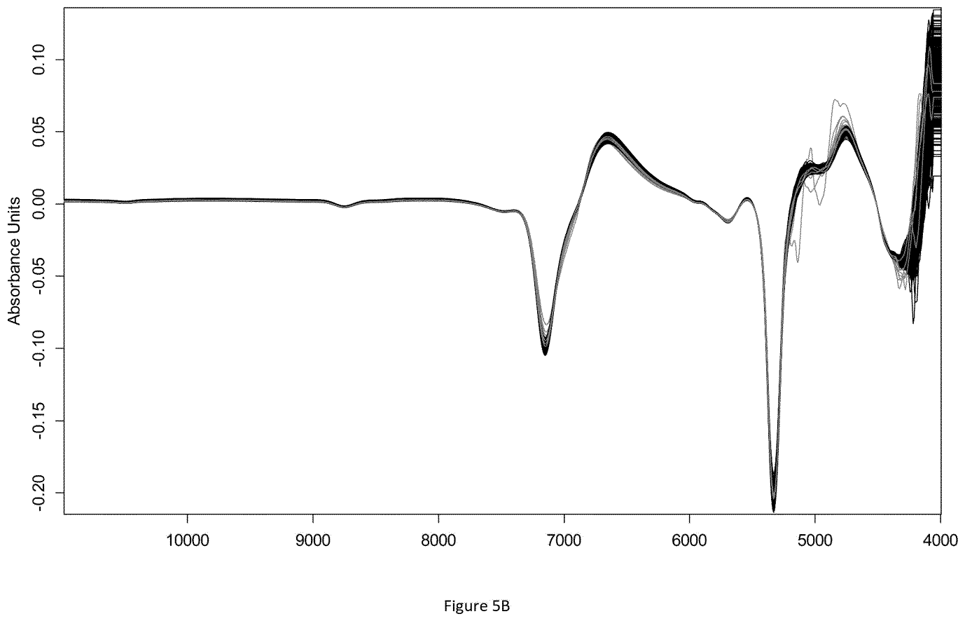

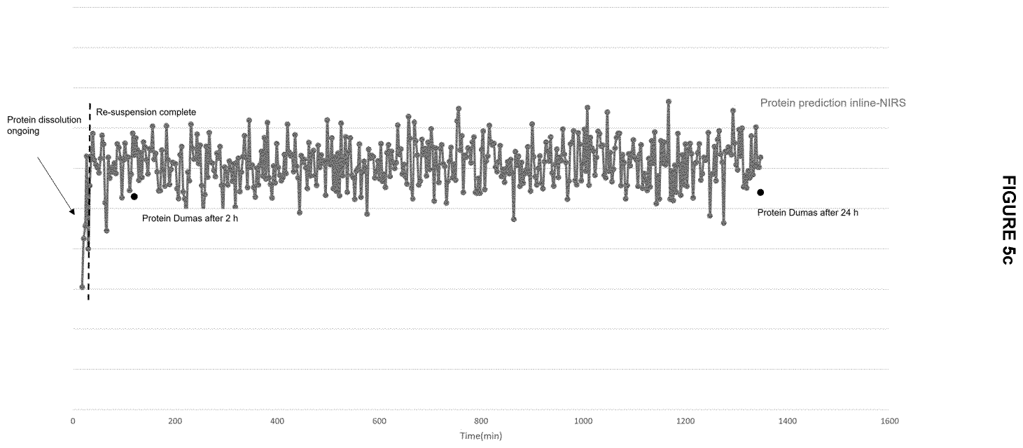

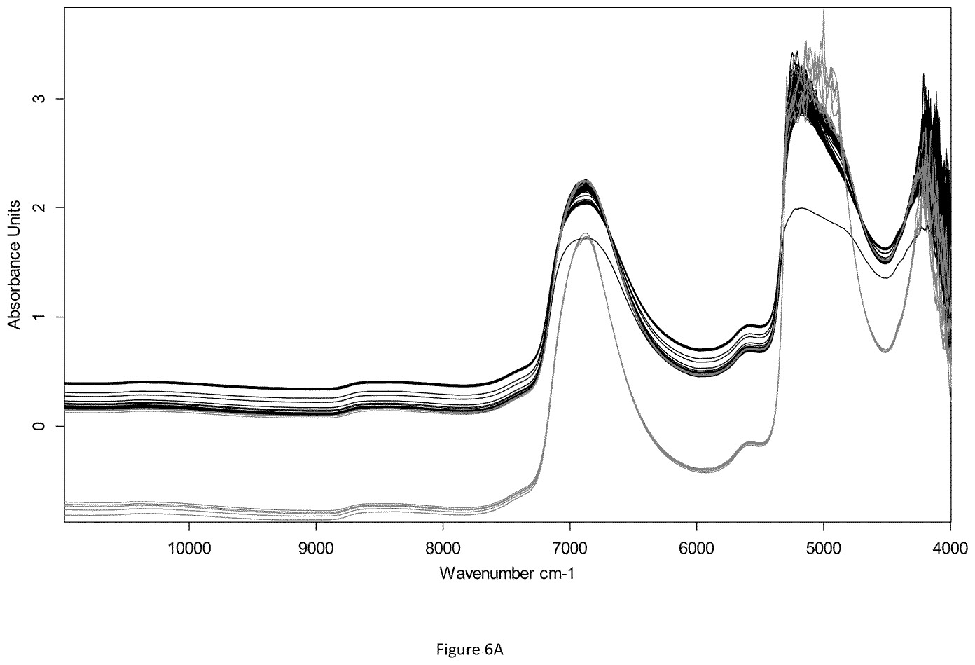

The in-line monitoring results are shown in FIGS. 4, FIG. 5 and FIG. 6 for resuspension reactions of three different Ig containing precipitate types (PPT A, Fr I+II+III, Fr II+III herein, respectively). In addition to the NIR raw spectra, spectra were pre-treated with 1^{st} derivative and vector normalization in order to emphasize spectral changes.

The results demonstrate that NIR models developed for at-line protein prediction work for in-line protein prediction during paste resuspension of alcohol (e.g. ethanol) precipitate types obtained from blood-derived plasma. The benefit of this approach is that it enables quantification of protein in a resuspension of a paste obtained from ethanol precipitation of blood-derived plasma without prior sample preparation, as is the case with current at-line and off-line procedures. Moreover, the methods enable monitoring of the progression of paste resuspension in real-time, potentially reducing cycle times between processing steps.

### Example 5—Description of NIR Measurement Setup for in-Line Ethanol (EtOH) in Plasma Fractionation on Manufacturing Scale

### NIR Spectrometer

The NIR measurements were conducted with a FT-NIR Matrix-F process spectrometer from Bruker Optics GmbH. The NIR process spectrometer can be used for spectroscopic analysis of liquids, suspensions and solids by transmission, diffuse reflectance and transflectance.

### NIR Probes

Transflectance was utilized to follow ethanol concentration and other matrix shifts during fractionation at manufacturing scale. Accordingly, NIR spectra in the scaled down model were recorded using a transflectance probe (IN271-02, 1 mm slit width) from Bruker.

NIR spectra were recorded at 3 min intervals using the following standardized instrument settings: pre-amplifier B (for product) or pre-amplifier A (for air background), 64 scans, spectral range 4′000-11′948 cm⁻¹, resolution 16 cm⁻¹.

### Software

Data acquisition was performed using the CMET from Bruker Optics GmbH.

Model optimization and calibration as well as prediction of datasets not included in the calibration data set were performed with the OPUS software package QUANT.

Qualitative analysis of the evolution of spectra due to changes in the sample matrix, especially changes in the ethanol concentration, were performed with the OPUS software package 3D.

### Chemometric Models or Multivariate Models

### Generation of Models Using QUANT

All models were generated based on the acquired spectra and the corresponding ethanol reference values using the Bruker QUANT software package. The optimum combination of the spectral ranges and the pre-treatment of the spectra was determined in the software's optimization tool. Cross validation was used for internal validation of the models.

### Model Quality Criteria

Similar criteria to that described above in Example 1 were considered when assessing the model quality and predictive ability of the different chemometric models or multivariate models.

### Example 6—at-Line EtOH Determination in Plasma Fractionation

### General NIR Models

The NIR models Model_{I+II+III} (developed using samples from plasma fractionation of Cohn Fraction (I+)II+III), Model_{IV} (developed using samples from plasma fractionation of Cohn Fraction IV) and Mod_{comp} (developed using samples from plasma fractionation of both Cohn Fraction (I+)II+III and Cohn Fraction IV)) were developed to facilitate the prediction of ethanol (EtOH) concentration in plasma fractionation steps.

Multivariate NIR models were generated by partial least squares (PLS) regression using the OPUS QUANT software package to process raw spectra of training samples in conjunction with the theoretical ethanol concentration.

Based on signal changes in the NIR loading plots the wavelength regions of approx. 9′400-5′448 cm⁻¹ were selected for Model_{comp}, Model_{I+II+III}, and Model_{IV} NIR model optimization.

NIR models were optimized with a pre-defined selection of spectral pre-treatments, e.g. 1^{st} derivative, vector normalization, min-max normalisation, straight line subtraction, and the combination of 1^{st} derivative and vector normalization.

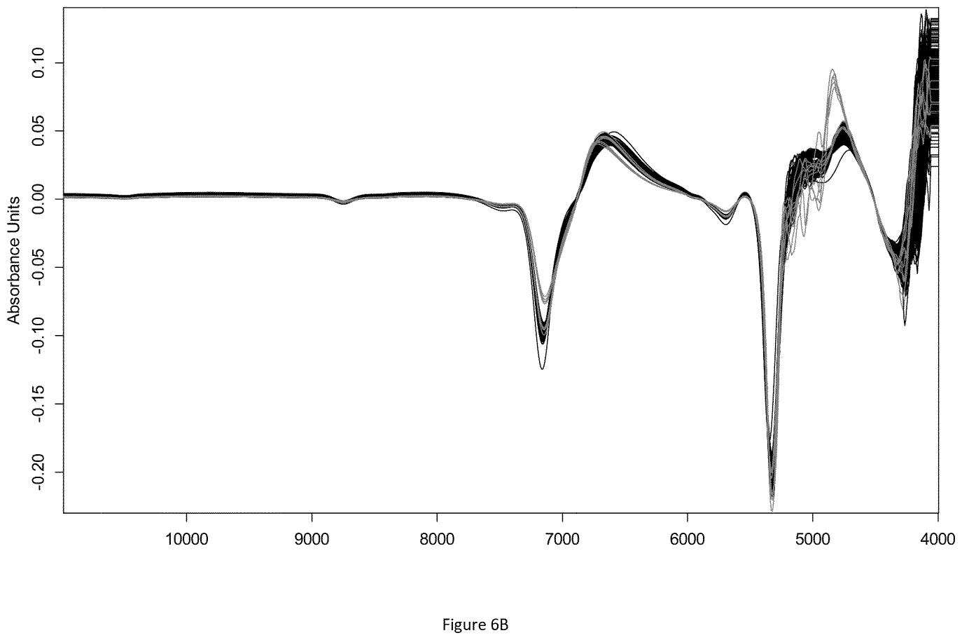

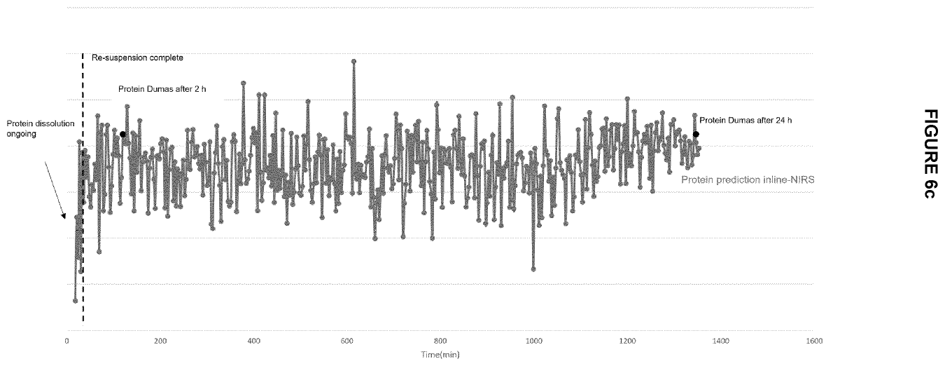

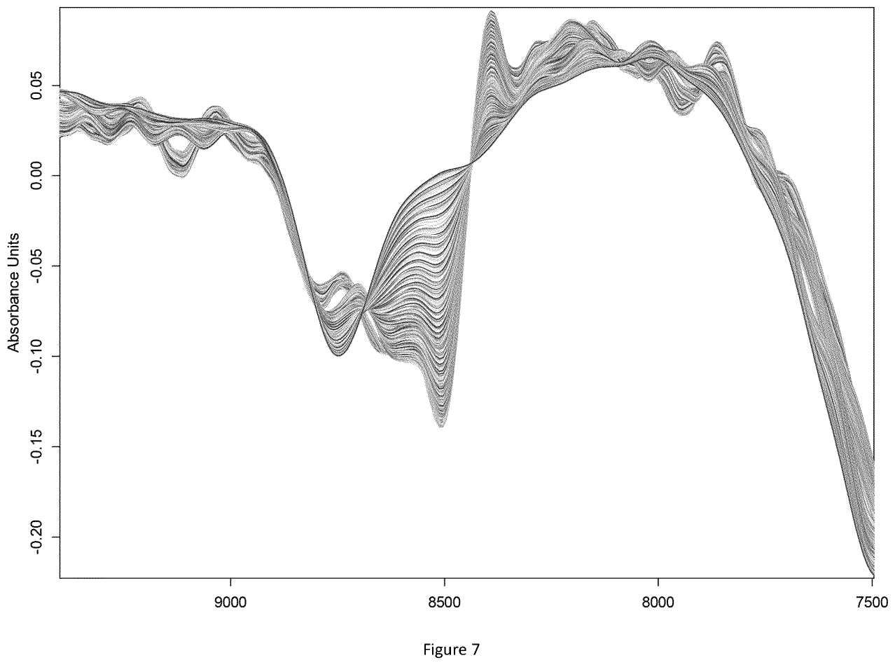

Model_{comp} is based on vector normalization and the first derivative as pretreatments and exhibits a root mean square error of cross validation (RMSECV) of 0.149 as well as a PLS rank of 10. An overlay of the pretreated spectra is shown in FIG. 7.

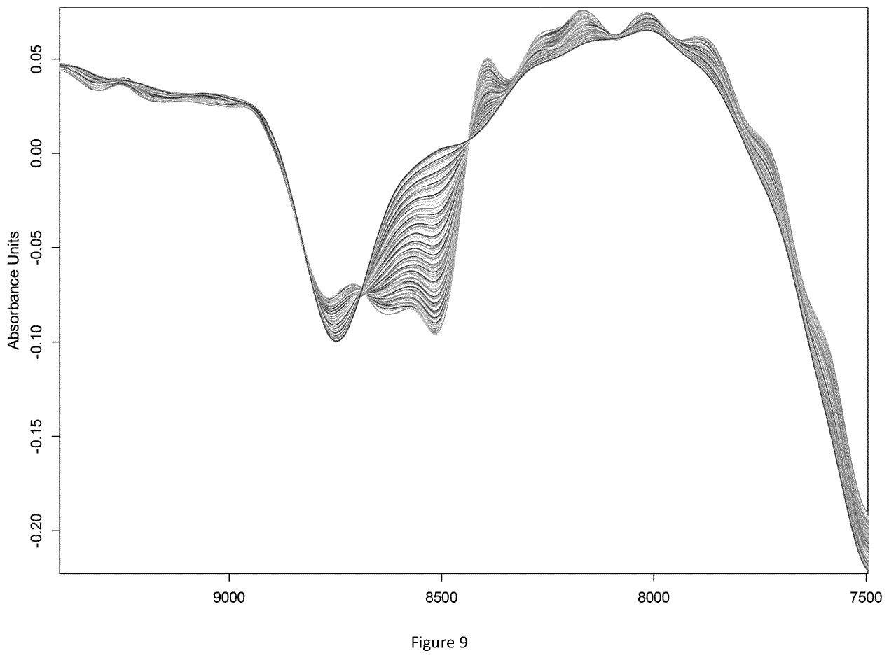

Model_{I+II+III} is based on vector normalization and the first derivative as pretreatments and exhibits a root mean square error of cross validation (RMSECV) of 0.0576 as well as a PLS rank of 7. An overlay of the pretreated spectra is shown in FIG. 9.

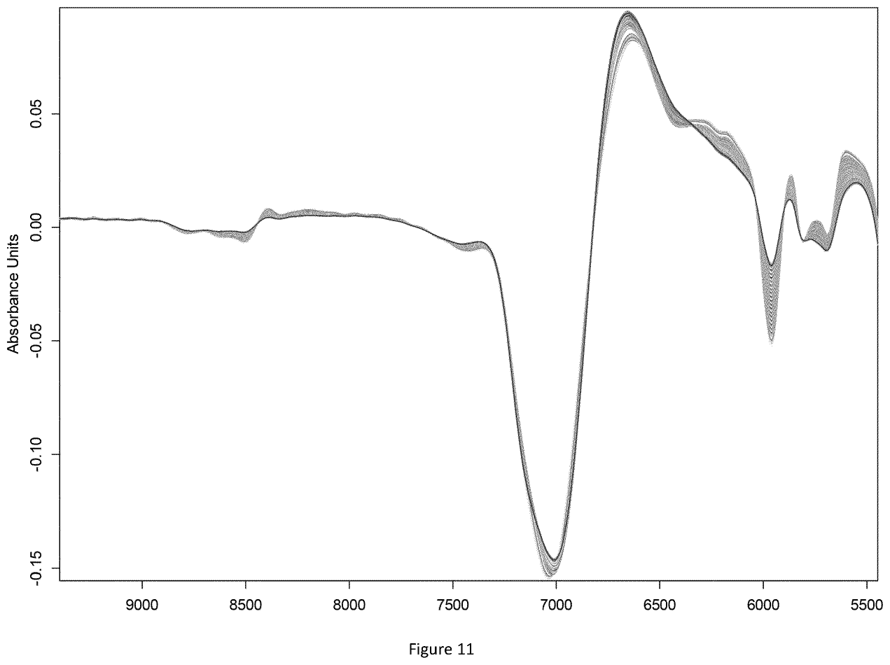

Model_{IV} uses vector normalization and the first derivative as pretreatments and exhibits a root mean square error of cross validation (RMSECV) of 0.0564 as well as a PLS rank of 9. An overlay of the pretreated spectra is shown in FIG. 11.

### Predictive Ability of Mod_{Comp}

Model_{comp} was utilized to predict the ethanol concentration in a manufacturing scale run of plasma fractionation of Cohn Fraction (I+)II+III and Cohn Fraction IV.

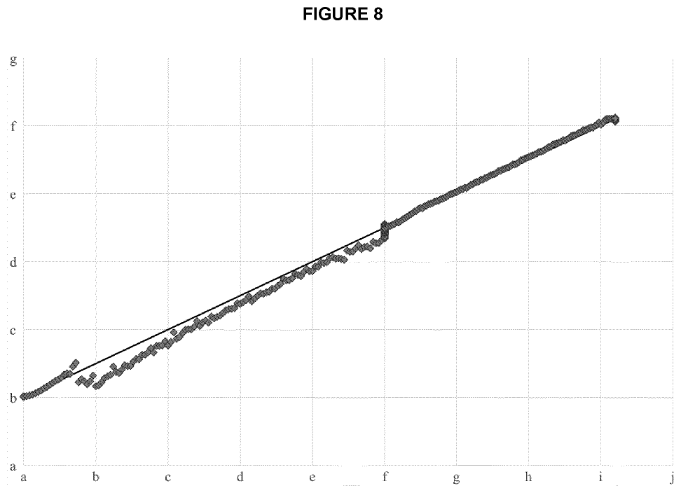

The theoretical ethanol concentration was calculated for each recorded spectrum based on the added amount i.e. weight of ethanol. Model_{comp} ethanol prediction aligned very closely to the theoretical concentration suggesting the model can accurately determine ethanol concentration in manufacturing scale plasma fractionation of Cohn Fraction (I+)II+III and Cohn Fraction IV (FIG. 8).

### Predictive Ability of Mod_{I+II+III}

Model_{I+II+III} was utilized to predict the ethanol concentration in a manufacturing scale run of plasma fractionation of Cohn Fraction (I+)II+III.

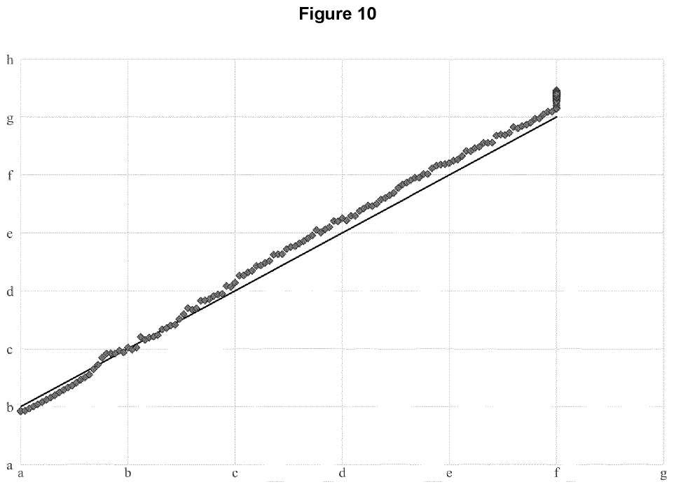

The theoretical ethanol concentration was calculated for each recorded spectrum based on the added amount i.e. weight of ethanol. Model_{I+II+III} ethanol prediction aligned very closely to the theoretical concentration suggesting the model can accurately determine ethanol concentration in manufacturing scale plasma fractionation of Cohn Fraction (I+)II+III (FIG. 10).

### Predictive Ability of Mod_{IV}

Model_{IV} was utilized to predict the ethanol concentration in a manufacturing scale run of plasma fractionation of Cohn Fraction IV.

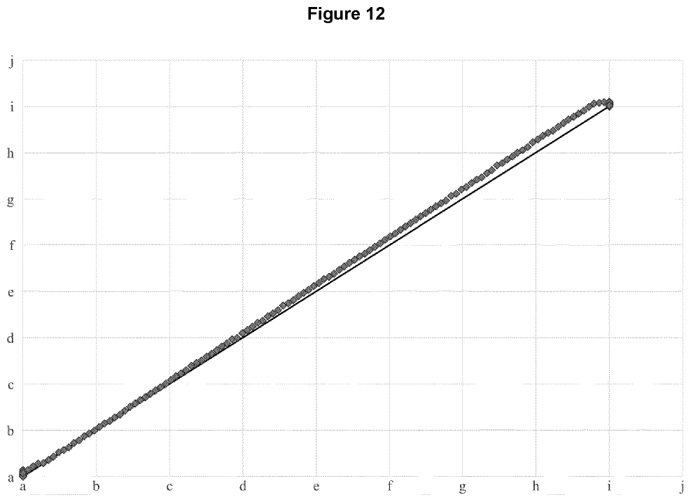

Theoretical ethanol was calculated for each recorded spectrum based on the added amount i.e. weight of ethanol. Model_{IV} ethanol prediction aligned very closely to the theoretical concentration suggesting the model can accurately determine ethanol concentration in manufacturing scale plasma fractionation of Cohn Fraction IV (FIG. 12).

### Example 7—at-Line Protein Determination in Albumin Precipitate Resuspension

Precipitate C (PPT C) or Precipitate V (PPT V) was resuspended in a double-jacketed vessel on a small scale (<1 L). Suspension samples were taken to prepare NIRS samples with different concentrations of PPT C or PPT V in vacuum flasks; these dilutions were prepared by mixing different amounts of suspensions, distilled water and filter aid. NIR transflectance spectra of these samples were collected in triplicates and utilized as the calibration set to build the model (Model_{Albresusp}).

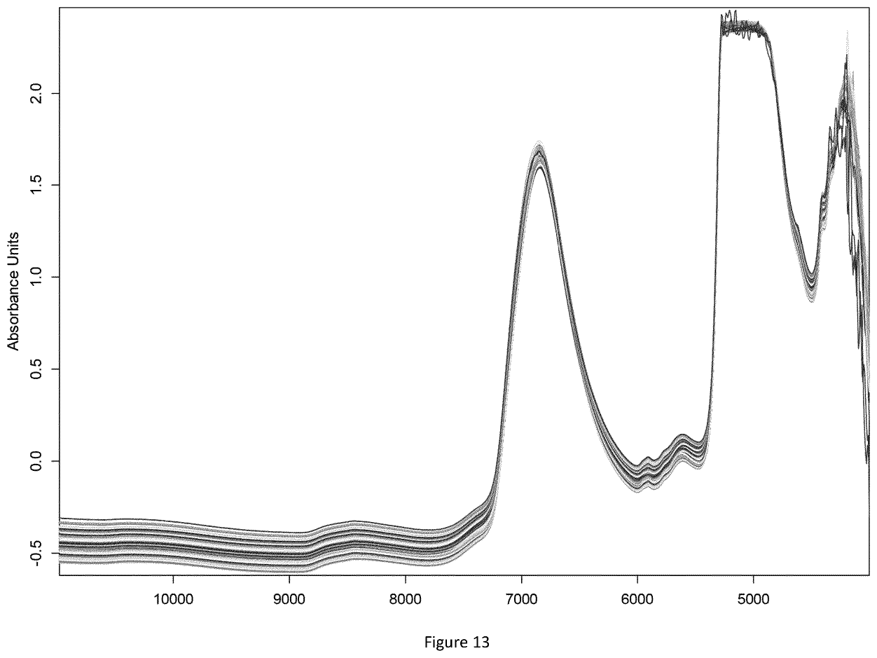

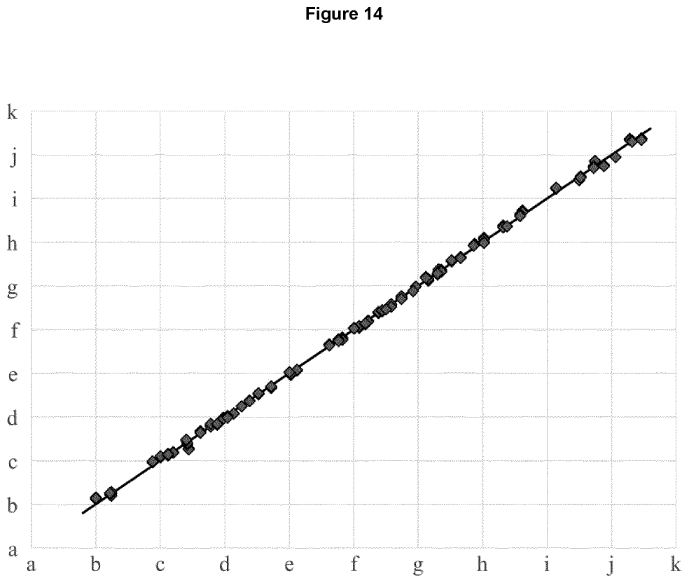

For the model to be used during resuspension (Model_{Albresusp}), as a spectral preprocessing method, a combination of 1st derivative and vector normalization delivered best results. FIG. 13 shows the NIR spectra of the training data set and FIG. 14 shows the resulting model can accurately determine protein concentration during resuspension of PPT C or PPT V.

Model_{Albresusp} uses vector normalization and the first derivative as pretreatments and exhibits a root mean square error of cross validation (RMSECV) of 0.284 as well as a PLS rank of 5.

TABLE 3
Properties and quality attributes of Model_{Albresusp}

| | Component | | | | | | |
|---|---|---|---|---|---|---|---|
| | range | #calibration | Spectral | | | | |
| Model | [g/kg] | spectra | ranges | RMSECV | R² | Rank | RPD |
| ModelAlbresusp | approx | 182 | 9002-7497.8 | 0.284 | 99.94 | 5 | 40.8 |
| | 45 g/kg (see | | 6032.2-5592.5 | | | | |
| | FIG. 14) | | | | | | |

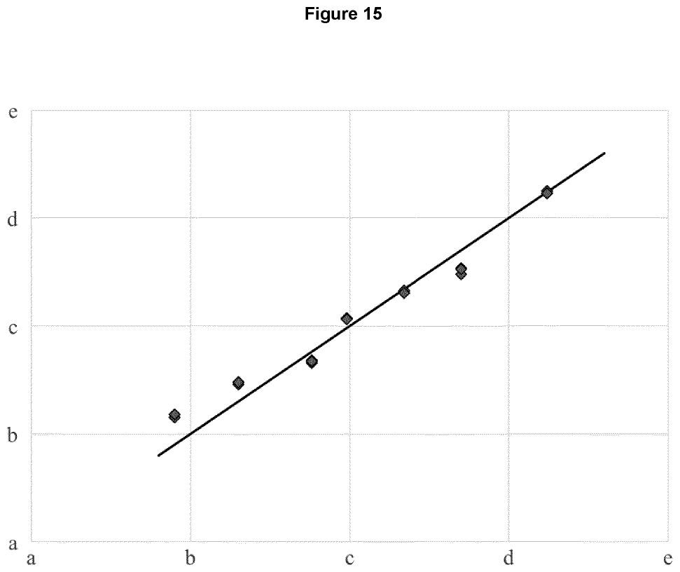

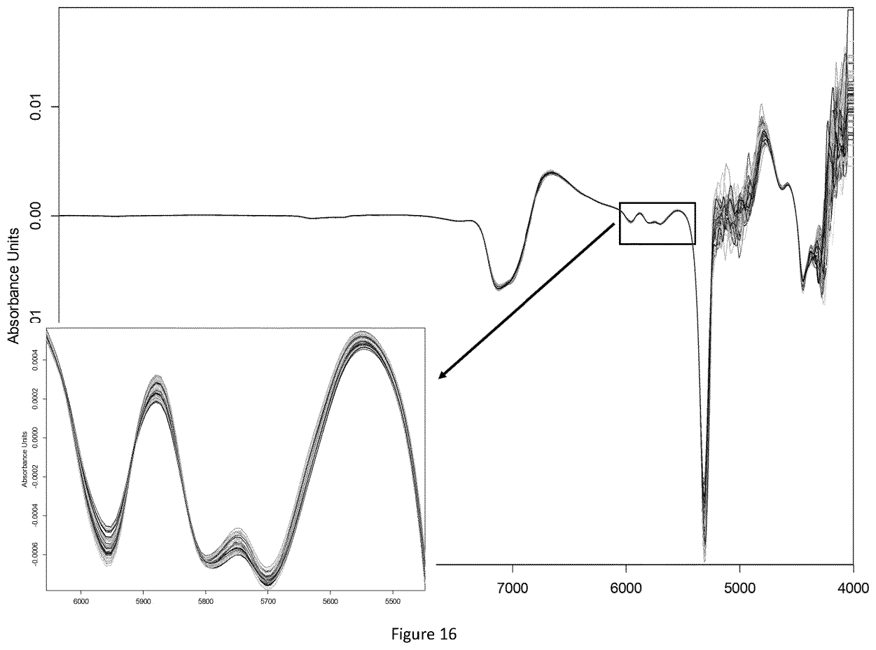

To be sure that the model makes reliable predictions for the protein concentration, it was tested against independent spectra of known corresponding reference protein value. In this case, 21 test spectra (7 samples in triplicate) were used to test the model, as shown in FIG. 15. The prediction of the independent test set resulted in a RMSEP of 0.733 g/kg. The predicted values of the Model_{Albresusp} correspond well with the actual protein concentration obtained with the Dumas assay. This confirms that Model_{Albresusp} can predict the protein concentration with good accuracy.

### Example 8—at-Line and in-Line EtOH Determination in Plasma Fractionation

NIR transflectance spectra of three different sample sets were acquired for this feasibility study:
1. Training set: PPT C (7 experimental series) or PPT V (3 experimental series) was resuspended on a small scale (<1 L). After resuspension was complete, a stepwise addition of ethanol was performed. The NIR probe was installed directly in the resuspension vessel (2 L glass reactor), spectra were recorded after each step of ethanol addition. A homogeneous mixture in the reactor was ensured by stirring for 10 minutes in between ethanol admixtures (350 rpm, overhead blade agitator). Spectra of these samples were collected in triplicates and utilized as the training set to build a model for ethanol concentration prediction. Spectral data acquisition was performed at 0±1° C.
2. Test set (at-line): Process samples were obtained from a pilot plant facility or the PAT lab. Samples were cooled to 0° C. (±1° C.) and prepared for spectra acquisition as described in Table 4 (test set at-line). At-line spectra of these samples were collected in triplicate. Those independent data sets were later utilized as the at-line test set to evaluate model performance.
3. Test set (in-line): Three independent albumin resuspension runs (2×PPT C, 1×PPT V) were conducted, where the near-infrared (NIR) probe was installed in the suspension vessel and spectra were acquired every minute in an in-line fashion.

For all analyzed samples the reference assay to determine the true ethanol concentration was performed via gas chromatography (GC).

TABLE 4
Data collection conditions employed for this feasibility
study. For each parameter it is listed to which
data set this specific condition applied.

| | Data sets | | |
|---|---|---|---|
| | Calibration | Test set | Test set |
| Parameter | set | (at-line) | (in-line) |
| Spectral resolution | 16 cm⁻¹ | | |
| No. of scans per | 64 | | |
| measurement | | | |
| Spectral wavenumber | 4000-11000 cm⁻¹ | | |
| range | | | |
| Preamplifier gain | A | | |
| (background) | | | |
| Preamplifier gain | B | | |
| (sample) | | | |
| Atmospheric | OFF | | |
| compensation | | | |
| Reference assay | Gas chromatography | | |
| Stirring time (before | n/a | 2 min | n/a |
| submerging probe) | | | |
| Stirring time (with | n/a | 1 min | n/a |
| probe submerged) | | | |
| Stirring time | 10 min | n/a | 1 min (continuous |
| (in-between | | | measurements) |
| measurements) | | | |
| Probe immersion depth | n/a | ~3 cm above | n/a |
| | | vessel bottom | |

To build a model, capable of ethanol concentration prediction, a partial least square (PLS) regression has been applied to selected spectral sets from the model training set samples. A total of N=34 samples have been included in the model training set.

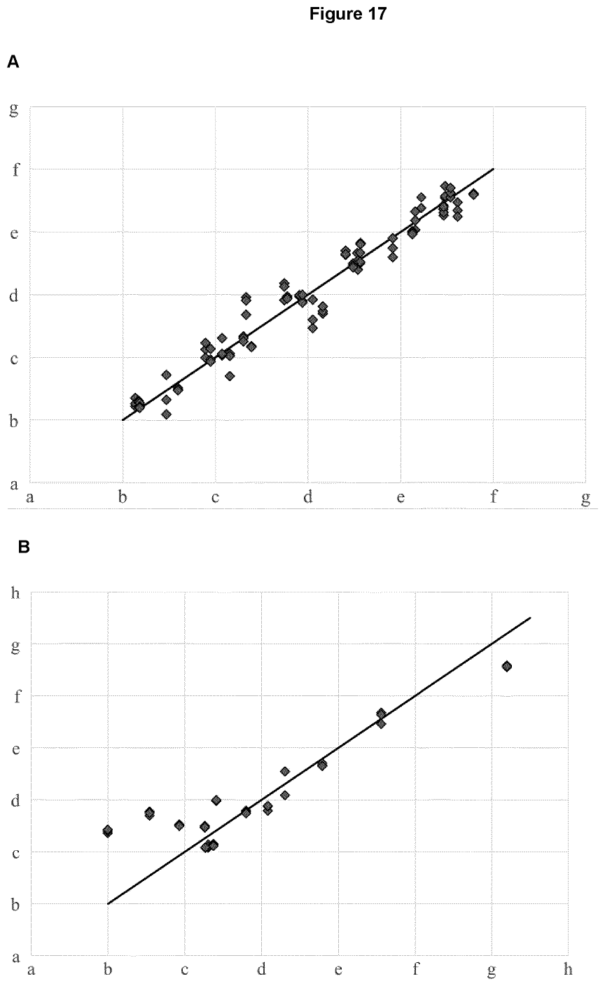

As a spectral preprocessing method, a combination of 1st derivative and vector normalization standard normal variate (SNV) delivered best results. In addition, spectral regions as outlined in Table 5 have been employed. The resulting preliminary ethanol quantification model, Model_{EtOH}, was then tested for its performance (see FIG. 17 (B) and Table 6).

TABLE 5
Detailed calibration characteristics of Model_{EtOH}

| | Spectral | Spectral | | |
|---|---|---|---|---|
| Model name | processing | regions [cm⁻¹] | RMSECV | Rank |
| Model_{EtOH} | 1^{st} derivative + | 9000-7992 | 0.217 | 7 |
| | Vector normalization | 6112-5392 | | |
| | (SNV) | 4656-4336 | | |

TABLE 6
Detailed characteristics of the predictive ability of Model_{EtOH}

| RMSEP | Bias | SEP | RPD | Offset | Slope |
|---|---|---|---|---|---|
| 0.584 | −0.175 | 0.416 | 2.28 | 2.564 | 0.695 |

Test Set (at-Line)

After a preliminary model has been established, 14 individual test set samples (11×PPT C, 3×PPT V) were quantified for their ethanol content, using the Model_{EtOH}. A comparison between the results from the QC reference assay and the ethanol concentration prediction are summarized in FIG. 17. With a RMSEP (root mean square error of prediction) of 0.584% w/w of ethanol in the suspension, the preliminary model provides a prediction of the ethanol content.

### Test Set (In-Line)

To follow the timely ethanol concentration change during resuspension of PPT C or PPT V an in-line NIRS scenario has been established on a laboratory scale (2×PPT C, 1×PPT V). During the resuspension of the PPT, the in-line NIR probe recorded a spectrum every minute, which was then immediately analyzed by the Model_{EtOH}. The resulting data was subsequently plotted (ethanol concentration [% w/w] against time [min]) and displayed in real-time on a connected computer. This enabled to follow the dissolution of ethanol from the PPT in a real-time fashion and thereby also allowed for an inference about the PPT's resuspension status and kinetics.

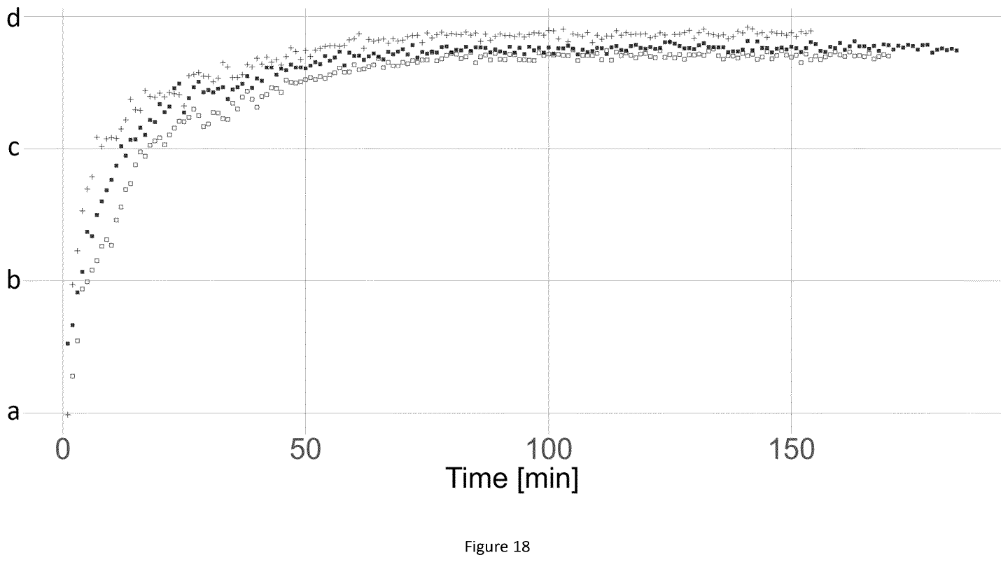

A total of three resuspension runs have been monitored in such fashion and are presented in FIG. 18.

### CONCLUSION

A NIR model capable of predicting the ethanol concentration at a given time during the dissolution and resuspension of PPT C and PPT V has been established. Even though a relatively small number of samples were employed for model calibration (N=34), a prediction (RMSEP=0.584% w/w ethanol) in the calibrated ethanol concentration range could be achieved, as demonstrated with individual test samples at-line (N=14).

In addition, a real-time monitoring of the ethanol concentration during PPT dissolution and resuspension was demonstrated by observing three runs with an in-line NIR probe followed by immediate spectral analysis. This enables real-time information concerning the ethanol concentration within the reactor at a given moment, allowing for inference concerning the PPT's dissolution state and the resuspension progression.

It will be understood that the invention disclosed and defined in this specification extends to all alternative combinations of two or more of the individual features mentioned or evident from the text or drawings. All of these different combinations constitute various alternative aspects of the invention.

1. A method for determining the concentration of an analyte in a sample obtained from processing of blood-derived plasma, the method comprising:
applying a light source in the near-infrared spectrum to a test sample obtained from processing of blood-derived plasma;
measuring reflectance, transmission, or transflectance of the test sample over a range of near-infrared wavelengths, thereby generating test wavelength spectra, and
comparing the test wavelength spectra with reference wavelength spectra obtained from reference samples having known concentrations of the analyte, to determine the concentration of the analyte in the sample.

2. The method of claim 1, wherein the test wavelength spectra are subjected to multivariate data analysis.

3. The method of claim 1, wherein
the comparing comprises comparing the test wavelength spectra to a reference data set in the form of a model generated using multivariate analysis of processed reference wavelength spectra of reference samples having known concentrations of the analyte.

4. A method for generating a model to determine the concentration of an analyte in a sample obtained from plasma processing, the method comprising:
providing training samples obtained from processing of blood-derived plasma, wherein the samples have known concentrations of the analyte;
applying a light source in the near-infrared spectrum to the training samples;
measuring the reflectance, transmission, or transflectance of the training samples over a range of near-infrared wavelengths, thereby generating training wavelength spectra;
selecting spectral regions of interest in the training wavelength spectra; and
generating a model by applying multivariate analysis to the spectra to provide a correlation with known concentration of the analyte,
thereby obtaining a model for determining the concentration of an analyte in a sample obtained from plasma processing.

5. The method of claim 2, wherein the multivariate analysis is selected from Partial least squares regression (PLS); PLS Discriminant Analysis (PLS-DA); Ordinary Least Squares (OLS) regression; MLR (multiple linear regression); OPLS (Orthogonal-PLS); SVM (support vector machines); GLD (general discriminant analysis); GLMC (generalized linear model); GLZ (generalized linear and non-linear model); LDA (Linear Discriminant Analysis); classification trees; cluster analysis; neural networks; and Pearson correlation.

6. The method of claim 3, wherein the model is a model generated using partial least squares (PLS) regression of processed wavelength spectra of samples having known concentrations of the analyte.

7. The method of claim 6, wherein the model generated is judged using one or more of the following statistical parameters:
Number of latent variables (PLS factors) in the model,
Bias,
RMSECV (root mean square error of cross validation),
RMSEP (root mean square error of prediction) for independent test samples,
R² (coefficient of determination), and
RPD (ratio of standard deviation and standard error of prediction) value.

8. The method of claim 1, wherein the method comprises applying at least one spectral pre-treatment to the wavelength spectra.

9. The method of claim 8 wherein the spectral pre-treatment is 1^{st} derivative, vector normalization, or a combination of both 1^{st} derivative and vector normalization.

10. The method of claim 1, wherein the mode of measurement is transflectance.

11. The method of claim 1, wherein the light source in the near-infrared range is applied to the test sample using a probe adapted to emit light having wavelengths in the near-infrared range.

12-15. (canceled)

16. The method of claim 1, wherein the analyte is total protein.

17. The method of claim 1, wherein the analyte is ethanol.

18. (canceled)

19. The method of claim 1, wherein the analyte is protein, and the concentration of protein in the reference samples is determined using the Dumas assay.

20. (canceled)

21. The method of claim 1, wherein the test sample comprising the analyte is a sample obtained from processing of blood-derived plasma obtained from human blood.

22. The method of claim 21, wherein the test sample is obtained or derived from the processing of blood-derived plasma that comprises fresh plasma, cryo-poor plasma, or cryo-rich plasma.

23. The method of claim 22, wherein the plasma is pooled plasma obtained from a number of donations and/or subjects.

24. The method of claim 1, wherein the test sample is obtained or derived from hyperimmune plasma.

25. The method of claim 1, wherein the test sample comprising the analyte is a resuspension of a precipitate or paste obtained from blood-derived plasma.

26. The method of claim 1, wherein the test sample comprising the analyte is a fraction selected from: Cohn Fraction I (Fr I), Cohn Fraction II+III (Fr II+III), Cohn Fraction I+II+III (Fr I+II+III), Cohn Fraction II (Fr II), Cohn Fraction III (Fr III), Cohn Fraction IV (Fr IV), Cohn Fraction V (Fr V), Kistler/Nitschmann Precipitate A, Kistler/Nitschmann Precipitate B, and Kistler/Nitschmann Precipitate C.

27-28. (canceled)

29. The method of claim 1, wherein the test sample is a turbid solution or suspension having Nephelometric Turbidity Units (NTU) selected from equal to or greater than 10 NTU, equal to or greater than 15 NTU, equal to or greater than 20 NTU, equal to or greater than 25 NTU, equal to or greater than 30 NTU, equal to or greater than 35 NTU, equal to or greater than 40 NTU, equal to or greater than 45 NTU, equal to or greater than 50 NTU, equal to or greater than 55 NTU, equal to or greater than 60 NTU, equal to or greater than 65 NTU, equal to or greater than 70 NTU, equal to or greater than 75 NTU, equal to or greater than 80 NTU, equal to or greater than 85 NTU, equal to or greater than 90 NTU, equal to or greater than 95 NTU, equal to or greater than 100 NTU, equal to or greater than 150 NTU, equal to or greater than 200 NTU, equal to or greater than 250 NTU, equal to or greater than 300 NTU, equal to or greater than 350 NTU, equal to or greater than 400 NTU, equal to or greater than 450 NTU, equal to or greater than 500 NTU, equal to or greater than 550 NTU, equal to or greater than 600 NTU, equal to or greater than 650 NTU, equal to or greater than 700 NTU, equal to or greater than 750 NTU, equal to or greater than 800 NTU, equal to or greater than 850 NTU, equal to or greater than 900 NTU, equal to or greater than 950 NTU, equal to or greater than 1,000 NTU, equal to or greater than 1,500 NTU, equal to or greater than 2,000 NTU, equal to or greater than 2,500 NTU, equal to or greater than 3,000 NTU, equal to or greater than 3,500 NTU, equal to or greater than 4,000 NTU, equal to or greater than 4,500 NTU, equal to or greater than 5,000 NTU, equal to or greater than 5,500 NTU, equal to or greater than 6,000 NTU, equal to or greater than 6,500 NTU, equal to or greater than 7,000 NTU, equal to or greater than 7,500 NTU, equal to or greater than 8,000 NTU, equal to or greater than 8,500 NTU, equal to or greater than 9,000 NTU, equal to or greater than 9,500 NTU, or equal to or greater than 10,000 NTU.

30-31. (canceled)

32. The method of claim 1, wherein any or all steps of the method are performed in-line, at-line, off-line, or on-line.
